(19) United States
(12) Patent Application Publication
(10) Pub. No.: US 2026/0098869 A1
(43) Pub. Date: Apr. 9, 2026

(54) METHODS AND SYSTEMS FOR RAPID DETECTION OF STROKE AND DETERMINATION OF ONSET VIA NEUROINFLAMMATION BIOMARKERS

(71) Applicant: The Regents of the University of Colorado, A Body Corporate, Denver, CO (US)

(72) Inventors: Robert Kowalski, Denver, CO (US); William Jones, Denver, CO (US)

(21) Appl. No.: 19/127,326

(22) PCT Filed: Nov. 3, 2023
(86) PCT No.: PCT/US2023/078715

§ 371 Date: May 5, 2025

Related U.S. Application Data
(60) Provisional application No. 63/382,374, filed on Nov. 4, 2022.
(60) Provisional application No. 63/385,939, filed on Dec. 2, 2022.

(51) Int. Cl. G01N 33/68

(52) U.S. Cl. CPC G01N 33/6893; G01N 33/6869; G01N 2333/46; G01N 2333/5412; G01N 2800/2871; G01N 2800/52

(57) ABSTRACT

Disclosed herein are methods, devices, and systems useful for identifying a subject having suffered a stroke and for identifying onset time of the stroke. In many embodiments, accurately determining a stroke and its onset allows for use of therapeutic drugs to alleviate symptoms that may not be available without these determinations. In many embodiments, the methods, devices, and systems are useful in identifying and analyzing IL-6 levels in the subject's blood. In some embodiments, the biomarker is detected using ELISA, SPR, molecular array, SiMoA, Ella, ECL.

### CROSS-REFERENCE TO RELATED APPLICATIONS

This application claims benefit of priority pursuant to 35 U.S.C. § 119(e) of U.S. provisional patent application Nos. 63/382,374 and 63/385,939, both entitled “Stroke Detection,” and filed on 4 Nov. 2022 and 2 Dec. 2022, respectively, which are hereby incorporated by reference in their entireties.

### GOVERNMENT LICENSE RIGHTS

This invention was made with Government support under #NIH 2674-12 awarded by the National Institute of Neurological Disorders and Stroke (NINDS). The Government has certain rights in the invention.

### FIELD

The disclosed processes, methods, and systems are directed to detecting stoke in a patient and determining its onset time.

### BACKGROUND

Neuroinflammation is ubiquitous in acute stroke and worsens outcome. However, the precise timing of the inflammatory response is unknown, hindering the design of acute anti-inflammatory therapeutic interventions. Accurate determination of stroke onset time is necessary in treating stroke victims, but methods for doing so are lacking.

What is needed are methods for rapidly assessing stroke in patients and determining time of onset.

### SUMMARY

Disclosed herein are methods and systems for identifying a subject suffering from a stroke and estimating the time of onset. In some embodiments, the method may comprise obtaining a blood sample from the subject, measuring a level of at least one biomarker in the blood sample, and comparing the biomarker level to a reference biomarker level to determine whether the subject is suffering from a stroke and determine an onset time of the stroke. In some embodiments, the biomarker is associated with inflammation, for example the biomarker is interleukin 6 (IL-6). In many embodiments, the obtaining the blood sample and measuring the level of the at least one biomarker does not occur at a hospital, in other embodiments, the obtaining and measuring may be at point of care, which may be in a hospital. In many embodiments, determining the onset time may be followed by administering an amount of a therapeutic drug effective to treat a stroke, for example an anti-clotting drug, in one embodiment tissue plasminogen activator (tPA). In many embodiments, a therapeutic drug effective to treat a stroke is administered if the onset time is determined to be less than or about 4.5 hours prior, by the measuring of the level of at least one biomarker. In many embodiments, the method may further include obtaining a second biological sample at a time after the first sample and measuring the at least one biomarker level in the second sample and comparing level of biomarker in the first sample and the second sample.

Also disclosed are devices and/or kits configured to receive and/or analyze a biological sample from a subject suspected of having a stroke, wherein the device and/or kit comprises an accepting unit configured to accept a volume of the biological sample, an identification unit configured to identify at least one biomarker in the biological sample, a measuring unit configured to measure the level of the at least one biomarker in the biological sample, and a display unit for displaying the level of the at least one biomarker. In some embodiments, the device and/or kit may further comprise a comparing unit for comparing the level of the at least one biomarker to a reference marker and may further indicate whether the subject has suffered a stroke and/or the time of onset. In some embodiments, the device and/or kit is a point-of-care device and/or kit and may be configured to determine the occurrence and onset time of the stroke about 20 minutes after receiving the blood sample. In many embodiments, the at least one biomarker is associated with inflammation, for one example interleukin 6 (IL-6).

Also disclosed are systems for identifying a patient suffering from effects of a stroke and determining an onset time of the stroke, comprising, a device for processing a biological sample of the subject suspected of having a stroke, a device for accepting the processed biological sample, a device for identifying at least one one biomarker in the sample, a device for measuring a level of the at least one biomarker, and a device for displaying the level to a user. In some embodiments, the system further includes an anti-clotting drug effective to treat a stroke in a subject suffering from same.

### BRIEF DESCRIPTION OF THE DRAWINGS

FIG. 1. Plasma IL-6 (interleukin-6) levels in first 133 min after stroke onset (values for SIMOA results are reduced by a factor of 10 for scale). †All values are median plasma neuroinflammatory factor concentrations, for patients with stroke and controls. ‡For ECL, the reference is 1.89 pg/mL (Todd et al). **For extracellular vesicle analyses, levels are expressed as arbitrary units of pixel density.

FIG. 2. Ultra-early neuroinflammatory factor levels in patients with acute stroke (Todd et al.).† *Plasma IL-6 levels measured by electrochemiluminescence immunoassay.

FIG. 3. Stroke-onset and 24-hour plasma IL-6 levels, in confirmed stroke patients MSU, mobile stroke unit; IL-6, interleukin 6. *Ischemic stroke, middle cerebral artery (MCA) territory. **Thalamic intracerebral hemorrhage (ICH). †Todd et al.

FIG. 4. Stroke-onset plasma protein and enzyme levels, in confirmed stroke patients* Plasma biomarker levels measured with single molecule array (SIMOA).

FIG. 5. Ultra-early plasma neuroinflammatory factor levels in acute stroke patients†* †All biomarkers are derived from plasma of blood collected from patients on the MSU immediately after stroke symptom onset. *Values are presented as median and full range. Blue represents acute stroke patient; red denotes control subject or reference value. Panel A) IL-6 concentrations quantified for by ELISA and ECL. For ECL, the reference is 1.89 pg/mL (Todd et al.) Panel B) Biomarkers derived from extracellular vesicles via heat-shock protein pulldown from plasma. Panel C) Biomarker concentrations quantified by SIMOA.

FIG. 6. Extracellular Vesicle-derived biomarkers levels at stroke onset and 24 hours in confirmed stroke patients—Neuroinflammatory factor concentrations derived from a subset of extracellular vesicles (EVs) in circulating blood of confirmed stroke patients treated on the mobile stroke unit (MSU). Panel A) computed tomography (CT) brain imaging for acute ischemic stroke (upper) and intracerebral hemorrhage (lower) patients included in EV analysis. Panel B) Proteome Profiler Human XL Cytokine Array (R&D, Minneapolis, MN #ARY022B), for one patient, including stroke-onset blood drawn on MSU, 24-hour blood drawn during acute hospitalization, and pooled blood for healthy adult controls. Panel C) comparison of stroke onset and 24-hour spot pixel density concentrations of neuroinflammatory factors. Panel D) heatmap of neuroinflammatory factors measured from EVs isolated from MSU, 24-hour and healthy control plasma.

FIG. 7. Extracellular vesicle-derived neuroinflammatory factors

FIG. 8. Comparison of stroke-onset plasma IL-6 concentration and time of day, patient age and NIHSS score. Comparison of plasma I1-6 levels at the time of stroke onset with possible mitigating factors. Panel A) Comparison of plasma IL-6 with time of day blood collected. Panel B) Comparison of plasma IL-6 levels and patient age. Panel C) Comparison of plasma IL-6 levels and initial National Institutes of Health Stroke Scale (NIHSS) score on the mobile stroke unit (MSU).

FIG. 9 is a bar graph showing neuroinflammatory markers: Extracellular Vesicles

FIG. 10 is a graph showing plasma IL-6 levels extrapolated from stroke onset to thrombolysis window.

FIG. 11 is a graph showing Ultra-early Plasma IL-6 levels for MSU confirmed acute stroke patients (N=17).

FIG. 12 is a graph showing Plasma IL-6 change in acute stroke in first 24 hrs in MSU patients (N=14).

FIG. 13 is the graph in FIG. 12 resized to focus on the levels of IL-6 over the first 270 minutes after stroke symptom onset.

FIG. 14 is a graph of Plasma IL-6 change in stroke mimics in first 24 hours (N=6).

### DETAILED DESCRIPTION

Disclosed herein are methods, devices, and systems useful in diagnosing stroke and determining onset times, also disclosed are acute immunomodulatory stroke therapies and diagnostic methods, devices, and systems useful in promoting improved outcomes.

Stroke is a leading cause of death and disability, affecting>12 million individuals worldwide each year. Neuroinflammation is a ubiquitous sequelae and has a bimodal effect on outcomes, inducing secondary injury early after ischemic brain insult or hemorrhage and beneficial neural protection and repair, hours or days later. Acute clinical stroke treatment presently focuses primarily on restoration of cerebral circulation, or mitigation of intracranial bleeding. The poststroke inflammatory cascade remains largely untreated. GFAP (glial fibrillary astrocytic protein), UCH-L1 (ubiquitin C-terminal hydrolase), and a constellation of other cytokines, chemokines and enzymes implicated in neuroinflammation are studied in stroke, including interleukins IL-6 ((interleukin-6), IL-17, TN Fα (tumor necrosis factor alpha) the enzyme MMP-9 (matrix metallopro-teinase-9), and CXCL4 (chemokine (C-X-C motif) ligand 4). Prevailing data suggest the inflammatory response occurs many hours after stroke. However, inflammatory factors have only been studied in the hours and days following acute stroke onset in humans, typically after the 3- to 4.5-hour window for stroke thrombolysis. Applicants sought to help fill this gap in knowledge earlier than previously possible by obtaining blood concurrent with brain imaging and neurological exam in the field, utilizing a prehospital treatment paradigm, the mobile stroke unit. Applicants hypothesized that the neuroinflammatory cascade begins within minutes after the onset of stroke symptoms, representing a possible target for ultra-early intervention.

Neuroinflammation is ubiquitous in acute stroke and worsens outcome. However, the precise timing of the inflammatory response is unknown, hindering the design of acute anti-inflammatory therapeutic interventions. Applicants sought to identify the onset of the neuroinflammatory cascade using a mobile stroke unit.

For data presented herein, blood was obtained, prehospital, on a mobile stroke unit. Outcomes measured included biomarker concentrations, modified Rankin Scale score, and National Institutes of Health Stroke Scale score, which are well known to those of skill in the art.

Disclosed below are results from one study analyzing forty-one adults, including 15 patients treated on the mobile stroke unit, and 26 healthy controls to establish biomarker reference levels. Median patient age was 74 (range, 36-97) years, 60% were female, and 80% White. Ten (67%) were diagnosed as stroke, with 8 (53%) confirmed and 2 likely transient ischemic attack or stroke averted by thrombolysis; 5 were stroke mimics. For strokes, median initial National Institutes of Health Stroke Scale score was 11 (range, 4-19) and 6 (75%) received tPA (tissue-type plasminogen activator). Blood was obtained a median of 58 (range, 36-133) minutes after symptom onset. Within 36 minutes after stroke, plasma IL-6 (interleukin-6), neurofilament light chain, UCH-L1 (ubiquitin C-terminal hydrolase L1), and GFAP (glial fibrillary acidic protein) were elevated by as much as 10 times normal. In EVs, MMP-9 (matrix metalloproteinase-9), CXCL4 (chemokine (C-X-C motif) ligand 4), CRP (C-reactive protein), IL-6, OPN (osteopontin), and PECAM1 (platelet and endothelial cell adhesion molecule 1) were elevated. Inflammatory markers increased rapidly in the first 2 hours and continued rising for 24 hours.

This study found the inflammatory cascade is activated as early as 36 minutes after stroke and progresses rapidly. This activation and progression is earlier than observed previously in humans and suggests injury from neuroinflammation may occur faster than had been surmised. Disclosed herein are methods and systems useful in better describing the stroke trajectory, more accurately identifying stroke, and estimating its onset. The disclosed methods, devices, and systems are useful in providing improved care and outcomes for subjects suffering from this devastating neurological disease.

Also disclosed are results from an expansion of the study above. In this analysis, forty-two adults were analyzed, including 28 patients treated on the MSU, and 14 controls. Nineteen (68%) MSU patients were diagnosed as stroke, with 17 (61%) confirmed and 2 likely TIA or stroke averted by thrombolysis; 9 were stroke mimics. For confirmed stroke patients, median age was 72 (range 36-87) years, 47% were female, and 82% white. Median initial NIHSS score was 7 (range 1-22) and 12 (71%) received intravenous thrombolysis with tPA or TNK. Blood was obtained a median of 57 (range 26-158) minutes after symptom onset.

The neuroinflammatory cascade was found to be activated within 36 to 133 minutes after stroke and progresses rapidly. This is earlier than observed previously in humans and suggests injury from neuroinflammation occurs faster than had been surmised.

### Definitions

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, or 0.05% of a given value or range. Whenever the term “about” or “approximately” precedes the first numerical value in a series of two or more numerical values, it is understood that the term “about” or “approximately” applies to each one of the numerical values in that series.

“Intravenous” administration refers to administering a drug (e.g., tissue plasminogen activator) into a vein of a patient, e.g., by infusion (slow therapeutic introduction into the vein).

The term “amelioration” as used herein refers to any improvement of a condition (for example stroke) of a patient suffering therefrom, by the administration of one or more treatments, drugs, and/or compositions, according to the present disclosure, to such patient or subject in need thereof. Such an improvement may be seen as a slowing down of the progression, or a cessation of the progression, of the patient's condition, a decrease in the frequency, duration, and/or severity of any symptom, and/or an increase in frequency or duration of symptom-free periods or a prevention of impairment or disability due to the condition.

A “biomarker” is a characteristic that is objectively measured and evaluated as an indicator of normal biological processes, pathogenic processes, or pharmacological responses to a therapeutic intervention. Biomarkers may be of several types: indicative, predictive, prognostic, or pharmacodynamics (PD). Indictive biomarkers indicate whether a subject is suffering from or has suffered a specific condition. Predictive biomarkers predict which patients are likely to respond or benefit from a particular therapy. Prognostic biomarkers predict the likely course of the patient's condition and may guide treatment. Pharmacodynamic biomarkers confirm drug activity and enable optimization of dose and administration schedule.

The term “biological sample” or “sample” refers to a specimen obtained from a subject for use in the present methods, and includes urine, whole blood, blood component (such as plasma, peripheral blood mononuclear cells, and/or extracellular vesicle), serum, saliva, sputum, tissue biopsies, cerebrospinal fluid, gut lavage, bronchioalveolar lavage, nasal lavage, and induced sputum.

The term “effective amount” refers to an amount of a compound of the invention or other active ingredient sufficient to provide a therapeutic or prophylactic benefit in the treatment or prevention of a condition or to delay or minimize symptoms associated with a condition. Further, a therapeutically effective amount with respect to a compound of the invention means that amount of therapeutic agent alone, or in combination with other therapies, that provides a therapeutic benefit in the treatment or prevention of a condition. Used in connection with a compound disclosed herein, the term can encompass an amount that improves overall therapy, reduces or avoids symptoms, causes, or progression of the condition, or enhances the therapeutic efficacy or synergies with another therapeutic agent.

The term “mammal” includes, but is not limited to, humans, mice, rats, guinea pigs, monkeys, dogs, cats, horses, cows, pigs, and sheep.

A “patient” or “subject” includes a mammal or animal, such as a human, cow, horse, sheep, lamb, pig, chicken, turkey, quail, cat, dog, mouse, rat, rabbit, or guinea pig. The animal can be a mammal such as a non-primate or a primate (e.g., monkey and human). In one embodiment, a patient is a human, such as a human infant, child, adolescent, or adult of any or indeterminant sex.

“Patient in need,” “subject in need,” or those “in need of treatment” include those already with existing condition (i.e., stroke, for example, without limitation, acute stroke), as well as those at risk of or susceptible to the condition. The terms also include human and other mammalian subjects that receive either prophylactic or therapeutic treatments as disclosed herein.

The terms “treat”, “treating” and “treatment” refer to eliminating, reducing, suppressing, or ameliorating, either temporarily or permanently, either partially or completely, a clinical symptom, manifestation or progression of an event or condition associated with the disorders and conditions described herein. As is recognized in the pertinent field, methods and drugs employed as therapies may reduce the severity of a given condition but need not abolish every manifestation of the condition to be regarded as useful. Similarly, a prophylactically administered treatment need not be completely effective in preventing the onset of a condition to constitute a viable prophylactic method or agent. Simply reducing the impact of a condition (for example, as disclosed herein, stoke, etc. and/or reducing the number or severity of associated symptoms, or by increasing the effectiveness of another treatment, or by producing another beneficial effect), or reducing the likelihood that the condition will reoccur and/or worsen in a subject, is sufficient. One embodiment of the invention is directed to a method for determining the efficacy of treatment comprising administering to a patient therapeutic treatment in an amount, duration, and repetition sufficient to induce a sustained improvement over pre-existing conditions, or a baseline indicator that reflects the severity of the particular disorder.

A stroke, as used herein refers to an ischemic or hemorrhagic condition with affecting a patient or subject. Occurrence of a stroke means either occlusion of a blood vessel in the brain in the case of ischemic stroke, or hemorrhage from a blood vessel in the brain in the case of hemorrhagic stroke. Onset time of a stroke means the time of occlusion of a blood vessel (ischemic stroke) or the time of hemorrhage, as reflected immediately or shortly thereafter by the presence of stroke symptoms. These symptoms may involve a range of deficits, including motor function, speech, vision, and cognitive function. Treating an ischemic stroke refers to, in the case of medical therapy in most cases, administering an anti-clotting or “thrombolytic” therapeutic compound, for example tissue plasminogen activator (tPA) or Tenecteplase (TNK). Both tPA and TNK are tissue plasminogen activators. tPA (alteplase) is typically administered via bolus and infusion. TNK is an alteplase variant with three amino acid substitutions and may be administered by a single bolus in acute stroke. Treatment for acute ischemic stroke may also include mechanical thrombectomy. Treatment for intracranial hemorrhage may involve medical and surgical interventions for alleviating or stopping the bleeding.

Interleukin 6 (IL-6), refers to a pleotropic cytokine, produced in response to infection and tissue insult.

A reference level, as used herein refers to an established level and/or range of a biomarker (for one example IL-6) in healthy adults. IL-6 levels may vary by age and in association with underlying, premorbid medical conditions such as arthritis and acute infection.

An anti-clotting drug, as used herein refers to tPA (alteplase) and TNK (Tenecteplase).

Tissue plasminogen activator (tPA), as used herein refers to recombinant fibrinolytic agent approved by the FDA for treatment of acute ischemic stroke. The mechanism of action of tPA is conversion of plasminogen to plasmin, which lyses fibrin and fibrinogen.

A point-of-care device, as used herein refers to a device that measures a target molecule in real-time in proximity to, or “at the bedside” of, a patient during evaluation and/or treatment of a particular condition.

### ABBREVIATIONS

CRP, C-reactive protein.
COPD indicates chronic obstructive pulmonary disease.
CXCL4, chemokine (C-X-C motif) ligand 4.
ECL, electrochemiluminescence immunoassay.
ED, emergency department.
ELISA, enzyme-linked immunosorbent assay.
EtOH, ethanol.
EV, extracellular vesicle.
GFAP, glial fibrillary acidic protein.
HSP, heat shock protein.
ICH, intracerebral hemorrhage.
IFN-γ, interferon gamma.
IL-6, interleukin-6.
LOS, length of stay.
MCA, middle cerebral artery.
MMP-9, matrix metalloproteinase-9.
MSU, mobile stroke unit.
NfL, neurofilament light chain.
NIHSS, National Institutes of Health Stroke Scale.
OPN, osteopontin.
PECAM1, platelet and endothelial cell adhesion molecule 1.
SIMOA, single molecule array.
TIA, transient ischemic attack.
tPA, tissue-type plasminogen activator.
TNFα, tumor necrosis factor alpha.
UCH-L1, ubiquitin C-terminal hydrolase.
WBC, white blood count.

Various biomarkers useful in determining whether a subject has suffered a stroke and estimating the time of onset are disclosed. In most embodiments, the biomarker is selected from a protein, a peptide, a ribonucleic acid, and fragments thereof. In many embodiments, the biomarkers may be one or more inflammatory factors, for example neuroinflammatory markers, including factors that indicate neurotoxicity and/or neuroprotection. In some embodiments, the disclosed biomarkers may be associated with a hyper-acute phase of stroke and/or the acute stroke course and recovery. In many embodiments, the biomarker may be selected from one or more of IL-6, neurofilament light chain (NfL), GFAP, CRP, MMP-9, CXCL4, UCH-L1, OPN, and PECAM1. In some embodiments, the biomarker may be selected from IL-6 and NfL. In many embodiments, the biomarker is IL-6.

Various methods of analyzing a biological sample for the presence of a biomarker are disclosed. In some embodiments, the method of analysis may be one or more of ECL, ELISA, Ella, Surface Plasmon Resonance, molecular array, SIMOA, PCR. In many embodiments, the analysis may include capturing the biomarker with an antigen binding protein or monoclonal antibody specific for the biomarker, wherein the capturing may be permanent or semipermanent. In some embodiments, the antigen binding protein or antibody may be immobilized on a substrate. In most embodiments, the analysis may result in a value that reflects the concentration of the biomarker in the biological sample. In other embodiments, the level of the biomarker may be relative to other biomarkers, for example a reference biomarker. In one embodiment, the biological sample is a blood sample, for example peripheral blood from a venipuncture, or a fraction thereof, for example peripheral blood mononuclear cells, serum, or extracellular vesicles, for example extracellular vesicles displaying one or more factors associated with stress conditions, for one example heat shock proteins.

The biological samples may be obtained shortly after stroke onset. In many embodiments, the biological samples are obtained within about 133 minutes after stroke onset, for example less than 150 min, 140 min, 130 min, 120 min, 110 min, 100 min, 90 min, 80 min, 70 min, 60 min, 50 min, 40 min, or 30 min, and greater than about 10 min, 20 min, 30 min, 40 min, 50 min, 60 min, 70 min, 80 min, 90 min, 100 min, 110 min, 120 min, 130 min, or 140 min. In many embodiments a second sample may be obtained at a second time. In many embodiments, the second sample is obtained more than about 30 min after the first sample, for example more than 10 min, 20 min, 30 min, 40 min, 50 min, 60 min, 70 min, 80 min, 90 min, 100 min, 200 min, 400 min, 500 min, 750 min, 1000 min, 1250 min, or 1500 min, and less than about 1750 min, 1500 min, 1250 min, 1000 min, 750 min, 500 min, 400 min, 300 min, 250 min, 100 min, 90 min, 80 min, 70 min, 60 min, 50 min, 40 min, 30 min, or 20 min after the first sample.

Confirmation of stroke in a subject may be associated with an elevated level of one or more of the disclosed biomarkers relative to at least one reference level. In some embodiments, the reference level may be predetermined from a study of healthy controls, for example subjects that have not suffered a stroke. Where the biomarker is IL-6, the reference level may be about 1.89 pg/mL.

The levels of biomarkers may increase over time after a stroke. In many embodiments, the level of biomarker may increase more than 1.5× over a period of time. In some embodiments, the level may increase more than 1.2×, 1.3×, 1.4×, 1.5×, 1.6×, 1.7×, 1.8×, 1.9×, 2.0×, 2.1×, 2.2×, 2.3v2.4×, 2.5×, 2.6×, 2.7×, 2.8×, 2.9×, 3×, 4×, 5×, 6×, 7×, 8×, 9×, or 10×, and less than about 15×, 12×, 10×, 9×, 8×, 7×, 6×, 5×, 4×, 3×, 2×, 1.9×, 1.8×, 1.7×, 1.6×, 1.5×, 1.4×, or 1.3×, and the period may be about 24 hours. In some embodiments, the period of time reflecting the increase in biomarker level may be less than 30 h, 29 h, 28 h, 27 h, 26 h, 25 h, 24 h, 23 h, 22 h, 21 h, 20 h, 19 h, 18 h, 17 h, 16 h, 15 h, 14 h, 13 h, 12 h, 11 h, 10 h, 9 h, 8 h, 7 h, 6 h, 5 h, 4 h, 3 h, or 2 h, and more than about 1 h, 2 h, 3 h, 4 h, 5 h, 6 h, 7 h, 8 h, 9 h, 10 h, 12 h, 15 h, 18 h, 20 h, 24 h, 28 h, or 30 h. In many cases, where the biomarker is IL-6, the level may increase during the hyper-acute (≤2 hours) and acute stage up to 24 hours after stroke.

Various disclosed methods include obtaining a second biological sample, after a period of time from obtaining a first biological sample, analyzing the first and second biological samples for the presence of one or more biomarkers, and determining concentration levels of the one or more biomarkers in each biological sample, and comparing the level of the one or more biomarkers in the first biological sample to the level of the one or more biomarkers in the second biological sample, wherein a change in biomarker level may be useful in determining the occurrence of a stroke and/or determining its onset, and wherein the time between obtaining the first and second biological samples is within the 4.5 hr cutoff for administering an anti-clotting agent. In some embodiments, the biomarker may be one or more of IL-6, neurofilament light chain, GFAP, CRP, MMP-9, CXCL4, UCH-L1, OPN, and PECAM1. In some embodiments the biomarker is selected from IL-6, NfL, and combinations thereof. In many embodiments the second biological sample may be collected 10 or more minutes after the first, for example, more than about 5 min, 6 min, 7 min, 8 min, 9 min, 10 min, 15 min, 20 min, 25 min, 30 min, 40 min, 50 min, 60 min, 70 min, 80 min, 90 min, 120 min, 180 min and 240 min and less than about 270 min, 240 min, 200 min, 180 min, 160 min, 140 min, 130 min, 120 min, 110 min, 100 min, 90 min, 80 min, 70 min, 60 min, 50 min, 45 min, 40 min, 35 min, 30 min, 25 min, 20 min, 15 min, 10 min, 9 min, 8 min, 7 min, or 6 min after collecting the first biological sample. In these embodiments, the change in biomarker level in the case of IL-6 is positive. The change in biomarker levels may be an increase over time of about 1% and 1000%, for example greater than about 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 60%, 70%, 80%, 90%, 100%, 110%, 120%, 130%, 140%, 150%, 200%, 250%, 300%, 350%, 400%, 450%, 500%, 550%, 600%, 650%, 700%, 750%, 800%, 850%, 900%, or 950%, and less than about 1000%, 950%, 900%, 850%, 800%, 750%, 700%, 650%, 600%, 550%, 500%, 450%, 400%, 350%, 300%, 250%, 200%, 150%, 140%, 130%, 120%, 110%, 100%, 90%, 80%, 70%, 60%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 1⁹%, 18%, 17%, 16%, 15, 1⁴%, 13%, 12%, 11%, 10%, 9%, 8%, 7%, 6%, 5%, 4%, and 3%.

In some embodiments, stroke may be confirmed when the IL-6 level may be from about 4.0 to about 40 pg/mL, for example 6.0 to about 20 pg/ml, for one example about 8 pg/ml. In some embodiments, the confirmatory IL-6 level may be greater than about 6.0 pg/mL, 6.1 pg/mL, 6.2 pg/mL, 6.3 pg/mL, 6.4 pg/mL, 6.5 pg/mL, 6.6 pg/mL, 6.7 pg/mL, 6.8 pg/mL, 6.9 pg/mL, 7.0 pg/mL, 7.1 pg/mL, 7.2 pg/mL, 7.3 pg/mL, 7.4 pg/mL, 7.5 pg/mL, 7.6 pg/mL, 7.7 pg/mL, 7.8 pg/mL, 7.9 pg/mL, 8.0 pg/mL, 8.1 pg/mL, 8.2 pg/mL, 8.3 pg/mL, 8.4 pg/mL, 8.5 pg/mL, 8.6 pg/mL, 8.7 pg/mL, 8.8 pg/mL, 8.9 pg/mL, 9.1 pg/mL, 9.2 pg/mL, 9.3 pg/mL, 9.4 pg/mL, 9.5 pg/mL, 9.6 pg/mL, 9.7 pg/mL, 9.8 pg/mL, 9.9 pg/mL, 10.0 pg/mL, 10.1 pg/mL, 10.2 pg/mL, 10.3 pg/mL, 10.4 pg/mL, 10.5 pg/mL, 10.6 pg/mL, 10.7 pg/mL, 10.8 pg/mL, 10.9 pg/mL, 11 pg/mL, 11.1 pg/mL, 11.2 pg/mL, 11.3 pg/mL, 11.4 pg/mL, 11.5 pg/mL, 11.6 pg/mL, 11.7 pg/mL, 11.8 pg/mL, 11.9 pg/mL, 12 pg/mL, 13 pg/mL, 14 pg/mL, 15 pg/mL, 16 pg/mL, 17 pg/mL, 18 pg/mL, 19 pg/mL, 20 pg/mL, 21 pg/mL, 22 pg/mL, 23 pg/mL, 24 pg/mL, 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL, 40 pg/mL, and less than about 40 pg/mL, 39 pg/mL, 38 pg/mL, 37 pg/mL, 36 pg/mL, 35 pg/mL, 34 pg/mL, 33 pg/mL, 32 pg/mL, 31 pg/mL, 30 pg/mL, 29 pg/mL, 28 pg/mL, 27 pg/mL, 26 pg/mL, 25 pg/mL, 24 pg/mL, 23 pg/mL, 22 pg/mL, 21 pg/mL, 20 pg/mL, 19 pg/mL, 18 pg/mL, 17 pg/mL, 16 pg/mL, 15 pg/mL, 14 pg/mL, 13 pg/mL, 12 pg/mL, 11.9 pg/mL, 11.8 pg/mL, 11.7 pg/mL, 11.6 pg/mL, 11.5 pg/mL, 11.4 pg/mL, 11.3 pg/mL, 11.2 pg/mL, 11.1 pg/mL, 11 pg/mL, 10.9 pg/mL, 10.8 pg/mL, 10.7 pg/mL, 10.6 pg/mL, 10.5 pg/mL, 10.4 pg/mL, 10.3 pg/mL, 10.2 pg/mL, 10.1 pg/mL, 10.5 pg/mL, 10.0 pg/mL, 9.9 pg/mL, 9.8 pg/mL, 9.7 pg/mL, 9.6 pg/mL, 9.5 pg/mL, 9.4 pg/mL, 9.3 pg/mL, 9.2 pg/mL, 9.1 pg/mL, 9.0 pg/mL, 8.9 pg/mL, 8.8 pg/mL, 8.7 pg/mL, 8.6 pg/mL, 8.5 pg/mL, 8.4 pg/mL, 8.3 pg/mL, 8.2 pg/mL, 8.1 pg/mL, 8.0 pg/mL, 7.9 pg/mL, 7.8 pg/mL, 7.7 pg/mL, 7.6 pg/mL, 7.5 pg/mL, 7.4 pg/mL, 7.3 pg/mL, 7.2 pg/mL, 7.1 pg/mL, 7.0 pg/mL, 6.9 pg/mL, 6.8 pg/mL, 6.7 pg/mL, 6.6 pg/mL, 6.5 pg/mL, 6.4 pg/mL, 6.3 pg/mL, 6.2 pg/mL, or 6.1 pg/mL.

In many embodiments, the sensitivity of the disclosed confirmatory level may be about 79%, for example greater than about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, or 98%, and less than about 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, or 74%. In some embodiments, the specificity may be about 36%, for example greater than about 30%, 35%, 40%, 45%, 50%, 55%, 60%, 65%, 70%, 75%, 80%, 85%, or 90%, and less than about 95%, 90%, 85%, 80%, 75%, 70%, 65%, 60%, 55%, 50%, 45%, or 40%. In many embodiments, the sensitivity and specificity may be useful for estimating the stroke onset time in the 4.5-hour thrombolysis window.

### EXAMPLES

### Example 1—Methods

Disclosed herein are results from a prospective, proof-of-concept, cohort investigation of ultra-early blood- and extracellular vesicle-derived markers of neuroinflammation, cerebral insult and outcome in acute stroke. The research was approved by the University of Colorado (UC) IRB. Written informed consent was obtained from each study patient, or a legally authorized representative. The study included adult patients treated acutely for suspected stroke on the UC mobile stroke unit and subsequently admitted for acute care to the UC Hospital. Outcome was assessed at the time of acute hospitalization discharge. Outcome measures included the final clinical diagnosis, modified Rankin Scale and National Institutes of Health Stroke Scale scores. Blood was obtained on the mobile stroke unit and 24 hours later, and plasma was analyzed with enzyme-linked and electrochemiluminescense immunoassays, and heat-shock protein isolation of vesicles. For the main statistical analyses, subjects were dichotomized into those with confirmed stroke vs. healthy adult controls. Patient biomarker levels were compared with clinical reference standards and with median and individual values for study control patients, and for grouped plasma of healthy adult controls in extracellular vesicle analyses. Data were analyzed with SPSS, version 28.0 (Armonk, NY). This article follows the STROBE reporting guideline. The data that support the findings of this study are available from the corresponding author upon reasonable request.

### Supplemental Methods

### Study Population and Data Collection

Demographics, premorbid status, and stroke onset clinical characteristics were recorded from the electronic medical record. Premorbid data included history of stroke, and disability status with the modified Rankin Scale (mRS) score. Stroke data included the National Institutes of Health Stroke Scale (NIHSS) score obtained acutely on the mobile stroke unit (MSU) and upon arrival to the University of Colorado Hospital (UCH) emergency department (ED). Stroke onset clinical lab values included the complete blood count (CBC) with differential, basic and complete metabolic panel, and coagulation tests. Stroke treatment data included administration of recombinant tissue plasminogen activator (tPA), mechanical thrombectomy, and medical and surgical interventions for intracranial hemorrhage. Radiological data included computed tomography (CT) of head obtained on the MSU, CT in the ED, and CT and magnetic resonance imaging (MRI) obtained during acute hospitalization.

### Healthy Adult Controls

For the single molecule array (SiMoA) analyses, 21 healthy adult controls were used, all from a cohort of healthy college athletes enrolled in a separate study of traumatic brain injury. For the enzyme-linked immunosorbent assay (ELISA) analyses, a group of 5 older healthy adults was used. These were enrolled in a separate study of cognitive function. Informed written consent was obtained for all participants engaged in these two studies, which were approved by the Institutional Review Board of the University of Denver. Stroke history and cardiovascular risk factors were not available for these healthy controls.

### Biosample Collection and Processing

Peripheral blood was collected for each patient from an upper extremity venipuncture site on the MSU, during the acute pre-hospital stroke evaluation. The first tube of blood drawn was set aside for the research study. Approximately 2-5 mL of blood was obtained in an ethylenediaminetetraacetic acid (EDTA) tube which was transported to the hospital on the MSU at room temperature as is done with the clinical blood sample used for patient care, and then placed in a 4° C. refrigerator in the UCH emergency department (ED) upon arrival of the MSU to UCH. The blood was then transported to the Department of Neurosurgery (Graner lab) and processed. Aliquots of whole blood, plasma and peripheral blood mononuclear cells (PBMC) were prepared and stored in a −80° C. freezer.

### Plasma Quantification of Neuroinflammatory and Neuronal Biomarkers

Plasma levels of IL-6 were determined using a sandwich enzyme-linked immunosorbent assay (QUANTIKINE HS Elisa, #HS600C, R&D SYSTEMS) following manufacturer's instructions. Briefly, plasma samples were thawed on ice before being diluted one-third with the 15 supplied RD5-4 diluent and assayed against a standard curve with IL-6 concentrations ranging from 0.156 pg/mL to 10 pg/mL. All samples were tested in duplicate. Sample IL-6 concentrations were determined by non-linear regression from the standard curves using GRAPHPAD PRISM V8 (GRAPHPAD, La Jolla, CA).

The NEUROLOGY-4-PLEX A ASSAY was used, according to the manufacturer's instructions, on the single molecule array (SiMoA) platform (SR-XTM BIOMARKER DETECTION SYSTEM, QUANTERIX, MA) to measure plasma levels of neurofilament-light (NfL), UCH-L1 and GFAP. Briefly, plasma samples were thawed on ice then centrifuged 5 minutes at 10,000×g. The resulting supernatants were diluted one-fourth in provided sample diluent and loaded to a 96-well plate alongside standards and quality controls (one analog and one digital). The NF-light calibration curve ranged from 0.646 to 514 pg/mL and the GFAP from 9.63 to 8,405 pg/mL. Then antibody-coated magnetic beads and biotinylated detector were added to the wells. After a wash step, streptavidin-ß-galactosidase is added. The fluorescent signal values generated from the calibration curves of known concentrations were fitted to a 4-parameter logistic curve with 1/y2 weighting, and sample concentration were extrapolated by the QUANTERIX software. All samples were run in duplicates on one occasion.

The MSD S-plex proinflammatory panel 1 kit (MESOSCALE DISCOVERY, Rockville, MD) uses a sandwich immunoassay format coupled to signal-enhanced electrochemiluminescence (ECL) detection to achieve femtograms per milliliter sensitivity. This assay kit was used to measure plasma levels of the following cytokines with known association with stroke that then underwent additional analysis (IL-6, IL-17, IL-1β, TNFα and IFNγ). Briefly, plasma samples were thawed on ice then centrifuged 3 minutes at 2,000×g. The clarified supernatants alongside 8 standards were loaded onto a 96-well plate with integrated screen-printed carbon ink electrodes on the bottom of each well that are used as solid-phase supports for binding reactions. The assay kit was used according to the manufacturer's instructions. The plate was read on a MESO QUICKPLEX SQ 120 instrument, which measures the intensity of ECL emitted, which is proportional to the amount of analyte present in the sample.

### Extracellular Vesicle Isolation and Quantification of Neuroinflammatory Factors

The HSP pull down method was conducted with Vn96 (pep1, or ME-10; see also sequence 65 of U.S. Pat. No. 8,956,878) and the reverse analogue sequence (pep2; vn96 reverse see U.S. Pat. No. 9,493,810); both synthesized by Vivitide (Gardener, MA). Two hundred pL of plasma sample was centrifuged at 17,000×g for 10 minutes. Supernatant was transferred to anew tube and the remaining pellet discarded. Ten μL of HSPA peptides PEP1 and 10 μL of PEP2 were added to each sample (VIVITIDE ME-020P-KIT; VIVITIDE, Gardner, MA). Samples were rotated for 30 minutes, and then centrifuged at 17,000×g for 15 minutes. Supernatant was carefully removed and the pellets were resuspended with 1 mL of PBS. After resuspension, 500 μL of additional PBS was added to the tubes to fill the remaining volume. Samples were then centrifuged at 17,000×g for 5 minutes. Supernatant was carefully removed and PBS resuspension was repeated for a total of two washes. After removing the PBS supernatant, 100 μL of RIPA (RADIO-IMMUNE PRECIPITATION ASSAY) buffer (SIGMA-ALDRICH, St Louis, MO, R0278-50 ML) and 100 μL of nuclear extraction reagent (NER) from NE-PER NUCLEAR AND CYTOPLASMIC EXTRACTION KIT (THERMO SCIENTIFIC, Rockford, IL #78833) were added to each pellet. Each sample was resuspended in solution, vortexed for 5-10 seconds, then rotated for 15 minutes. Samples were then used to probe the PROTEOME PROFILER HUMAN XL CYTOKINE ARRAY (R&D, Minneapolis, MN #ARY022B). Manufacturer's protocol for the PROTEOME PROFILER HUMAN XL CYTOKINE ARRAYS was followed. However, the provided chemiluminescence reagents were substituted with developer from the SUPER SIGNAL WEST FEMTO KIT (THERMO SCIENTIFIC, 34095). Cytokine arrays were imaged using the FLUORCHEMQ MULTI IMAGE 3 (ALPHA INNOTECH/PROTEINSIMPLE, Wallingford, CT). Images were optimized using Photoshop and spots were quantified using NIH ImageJ (imagej.nih.gov/ij/). Heatmaps were generated using MICROSOFT EXCEL (MICROSOFT 365).

### Statistical Analysis

Patients were dichotomized into those with confirmed stroke vs. patients without confirmed stroke, and patients with confirmed stroke vs. healthy controls. Two patients diagnosed with stroke symptoms, but likely either a transient ischemic attack (TIA) or averted by tPA, were included with the non-stroke group. Patient premorbid disability status (mRS), clinical acute stroke status (NIHSS), and discharge status (mRS, NIHSS) were recorded individually by patient and as median and range. Stroke neuroninflammatory factors were evaluated by concentration in plasma and by arbitrary units of pixel density in association with extracellular vesicles. Biomarkers were recorded individually by patient, and as median and range.

### Example 2—Results

The study included 41 adult individuals. 15 patients with acute stroke symptoms treated on the University of Colorado mobile stroke unit were enrolled, and ultra-early blood samples were obtained. Median age was 74 years (range, 36-97), 60% were female, and 80% White. Ten patients (67%) were diagnosed as stroke, with 8 (53%) confirmed by clinical neurological examination and/or neuroimaging (Table 1; Table 2). The study included 26 healthy adults, and 1 pooled group of controls, for plasma and extracellular vesicle analyses.

Peripheral venous blood was obtained at the patients' homes at a median of 58 (range, 36-133) minutes after stroke symptom onset for all 8 confirmed stroke cases. Blood samples were obtained prior to administration of tPA and was drawn between 11:11 AM and 6:43 PM, on all days of the week except Wednesday.

### Plasma Neuroinflammatory Factor Levels

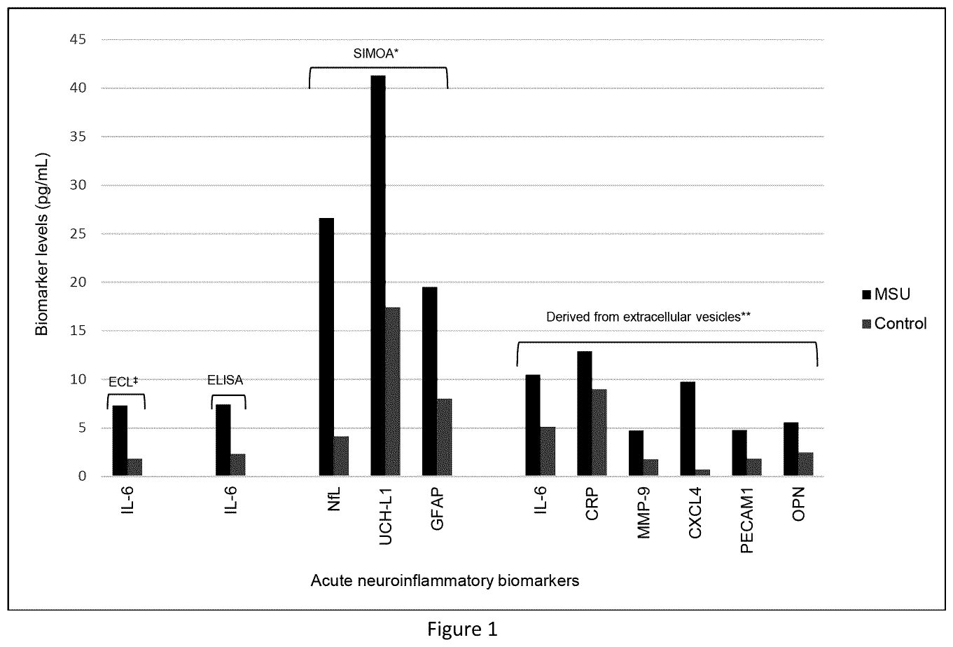

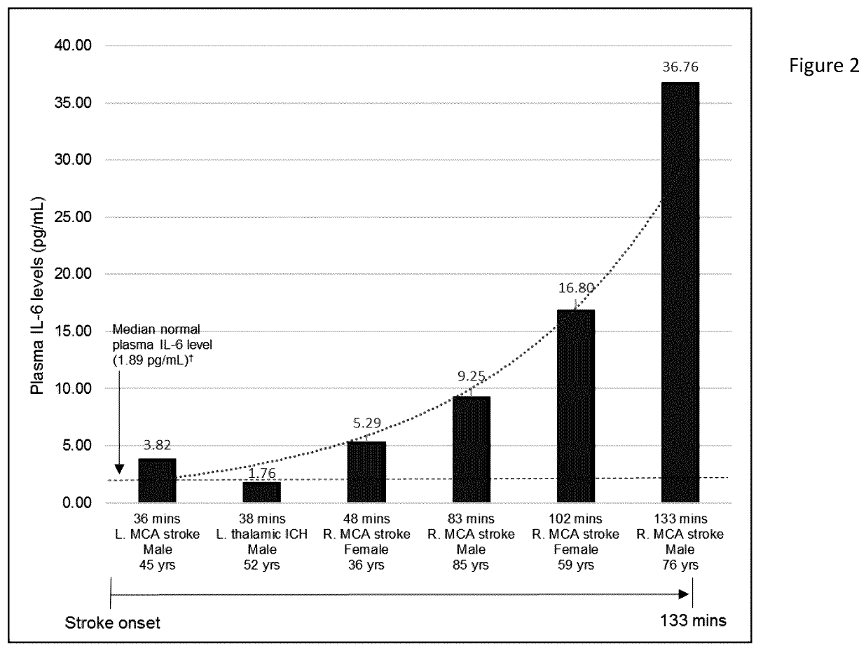

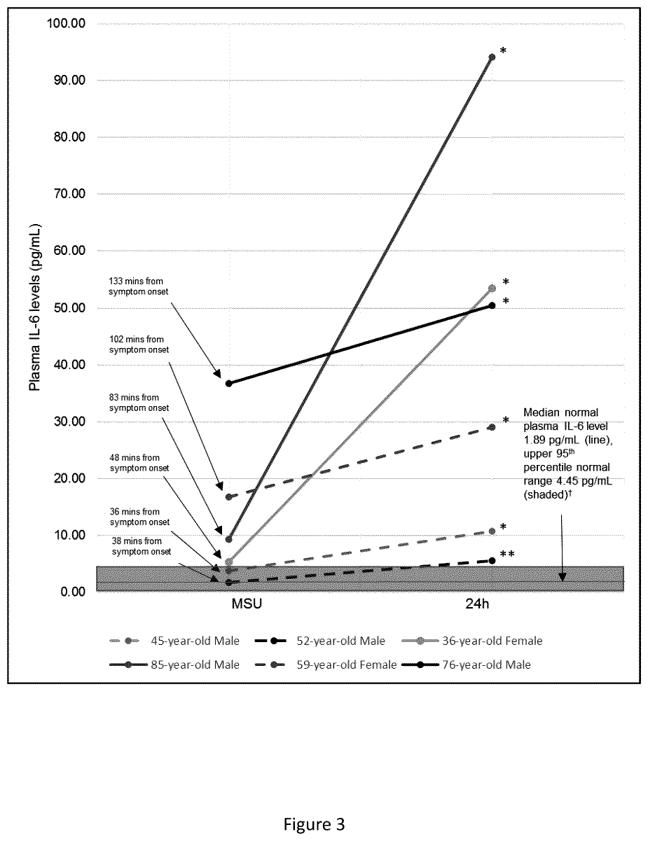

Among patients with confirmed stroke, plasma concentrations of IL-6, neurofilament light chain, UCH-L1, and GFAP were abnormally elevated above reference or healthy control levels (reference levels may be reported in published literature and/or in materials produced by laboratories, and healthy control levels may be those measured by Applicants using the same assays applied to the stroke patients in this study) by 36 minutes after stroke symptom onset, and in blood samples taken during the first 133 minutes after stroke (FIGS. 1 and 2; FIGS. 3 through 5). These biomarkers were as much as 10 times normal levels (Tables 3 through 7). Plasma concentrations of pro-inflammatory cytokines IL-17A, IL-1β, TNFα, and IFN-γ (interferon gamma) were in the normal range during this time period (Table 4). Median plasma IL-6 concentration, measured by electrochemiluminescence, was about 4 times higher in confirmed stroke patients (7.27 pg/mL, range, 1.76-36.76), compared with a median 1.89 pg/mL (range, 0.3-5.0) for healthy adults. 12, 13 Follow-up blood samples were obtained at 24 hours during acute hospitalization for 6 (750) of confirmed stroke patients. Plasma IL-6 levels increased between 1.7- and 10.2-fold in all patients during this 24-hour time period (FIG. 3). Plasma IL-6 was also elevated when assessed with enzyme-linked immunosorbent assay (ELISA). Plasma proteins neurofilament light chain, UCH-L1, and GFAP were quantified with single molecule array in 6 confirmed stroke patients (4 acute ischemic stroke, 2 intracerebral hemorrhage), and 21 adult healthy controls. All were elevated in stroke patients compared with controls, both in the minutes after stroke onset and at 24 hours (Tables 6 and 7).

TABLE 1
Baseline Demographic and Clinical Characteristics
of Patients Treated on Mobile Stroke Unit

| | | | |
|---|---|---|---|
| | All patients | Confirmed | Stroke |
| Patient characteristics | (N = 15) | stroke (N = 8) | mimic*(N = 7) |

Demographics

| | | | |
|---|---|---|---|
| Age (years at stroke symptom onset) | 74 (36-97) | 67 (36-86) | 74 (40-97) |
| Sex (female) | 9 (60) | 3 (37) | 6 (86) |

Race

| | | | |
|---|---|---|---|
| White | 12 (80) | 6 (75) | 6 (86) |
| Black | 2 (13) | 1 (12) | 1 (14) |
| Hispanic | 1 (7) | 1 (12) | 0 (0) |

Premorbid characteristics
Modified Rankin Scale score

| | | | |
|---|---|---|---|
| 0. No symptoms | 9 (60) | 6 (75) | 3 (43) |
| 1. No significant disability | 3 (20) | 2 (25) | 1 (14) |
| 2. Slight disability | 0 (0) | 0 (0) | 0 (0) |
| 3. Moderate disability | 1 (7) | 0 (0) | 1 (14) |
| 4. Moderately severe disability | 2 (13) | 0 (0) | 2 (29) |
| 5. Severe disability | 0 (0) | 0 (0) | 0 (0) |
| Previous stroke | 4 (27) | 3 (37) | 1 (14) |

Acute clinical characteristics

| | | | |
|---|---|---|---|
| MSU NIHSS score | 6 (1-22) | 11 (4-22) | 6 (1-7) |
| 0-4. Minor | 5 (33) | 2 (25) | 3 (43) |
| 5-15. Moderate | 8 (53) | 4 (50) | 4 (57) |
| 16-20. Moderate to severe | 0 (0) | 0 (0) | 0 (0) |
| 21-42. Severe | 2 (13) | 2 (25) | 0 (0) |
| ED NIHSS score | 5 (0-29) | 7 (1-29) | 2 (0-11) |
| 0-4. Minor | 7 (47) | 2 (25) | 5 (71) |
| 5-15. Moderate | 5 (33) | 3 (37) | 2 (29) |
| 16-20. Moderate to severe | 1 (7) | 1 (12) | 0 (0) |
| 21-42. Severe | 2 (13) | 2 (25) | 0 (0) |
| Δ NIHSS score (MSU to ED) | −1 (−4 to +4) | +0.5 (−4 to +5) | −1 (−4 to +2) |
| Improved | 8 (53) | 3(37) | 5 (71) |
| Unchanged | 1 (7) | 1(12) | 0 (0) |
| Worsened | 6 (40) | 4 (50) | 2 (29) |

Etiology

| | | | |
|---|---|---|---|
| Confirmed acute stroke | 8 (53) | 8 (100) | N/A |
| Ischemic | 6 (40) | 6 (75) | N/A |
| Hemorrhagic | 2 (13) | 2 (25) | N/A |
| Unconfirmed stroke or mimic | 7 (47) | N/A | 7 (100) |
| TIA or stroke aborted by tPA | 2 (13) | N/A | 2 (29) |
| Seizure | 2 (13) | N/A | 2 (29) |
| Pulmonary embolism | 1 (7) | N/A | 1 (14) |
| COPD exacerbation | 1 (7) | N/A | 1 (14) |
| EtOH withdrawal | 1 (7) | N/A | 1 (14) |

Acute treatment

| | | | |
|---|---|---|---|
| Time from symptom onset to | 69 (35-835) | 58 (36-133) | 405 (35-835) |
| MSU stroke treatment, mins† | | | |
| Thrombolysis-IV tPA‡ (on MSU) | 5 (40) | 4 (50) | 1 (14) |
| Thrombolysis-IV tPA‡ (ED) | 2 (7) | 2 (25) | 0 (0) |
| Mechanical thrombectomy | 2 (13) | 2 (25) | 0 (0) |

Hospital course

| | | | |
|---|---|---|---|
| Hospital LOS, d | 4 (3-6) | 4 (4-6) | 4 (1-6) |

Data are N (%), median (range).
COPD indicates chronic obstructive pulmonary disease; ED, emergency department; EtOH, ethanol; LOS, length of stay; MSU, mobile stroke unit; NIHSS, National Institutes of Health Stroke Scale; TIA, transient ischemic attack; and tPA, tissue-type plasminogen activator.
*Includes one patient with suspected TIA, and 1 patient with a possible ischemic stroke aborted by tPA.
†Time of blood draw on MSU.
‡Alteplase

TABLE 2
Stroke onset characteristics of patients with confirmed stroke treated on mobile stroke unit

| | | | | Acute Clinical Evaluation | | | | | | | | | | | |
|---|---|---|---|---|---|---|---|---|---|---|---|---|---|---|---|
| | | | | Evaluation | | | | | | | | | | | |
| | | | | timing | | | | | | | | | | | |
| | | | | Minutes | | | | MSU clinical presentation | | | | | | | Radiological |
| | | | | from | | | | Gaze | | | | | | | findings† |
| Patient characteristics | | | | stroke | | | | deviation | | | | | | | Non-contrast |
| Patient | | | Pre- | symptom | Stroke onset | | | Visual | | | | | | | head CT |
| no | | | morbid | onset on | NIHSS | | | field | Facial | Weak- | Sensory | Apha- | Dysar- | Inatten- | (MSU) and |
| [symbol] | Sex | Age | mRS | MSU | MSU | ED | Δ‡ | loss | paralysis | ness | loss | sia | thria | tion | Head CTA |
| 1 | M | 85 | 0 | 83 | 15 | 20 | +5 | X | X | X | X | | X | | R. M1 |
| | | | | | | | | | | | | | | | occlusion, loss |
| | | | | | | | | | | | | | | | gray-white |
| | | | | | | | | | | | | | | | differentiation |
| | | | | | | | | | | | | | | | R. insula |
| 2 | F | 36 | 0 | 48 | 22 | 26 | +4 | | | X | X | X | X | | R. M2 |
| | | | | | | | | | | | | | | | occlusion |
| 3 | F | 59 | 0 | 102 | 11 | 6 | −5 | | X | X | X | | X | X | R. M2 |
| | | | | | | | | | | | | | | | superior |
| | | | | | | | | | | | | | | | division |
| | | | | | | | | | | | | | | | occlusion, R. |
| | | | | | | | | | | | | | | | MCA hypo- |
| | | | | | | | | | | | | | | | attenuation |
| 4 | M | 76 | 1 | 133 | 4 | 5 | +1 | X | | X | | X | | X | R. M2 |
| | | | | | | | | | | | | | | | occlusion, |
| | | | | | | | | | | | | | | | encasement |
| | | | | | | | | | | | | | | | by petroclival |
| | | | | | | | | | | | | | | | meningioma |
| 5 | M | 78 | 1 | 40 | 21 | 29 | +8 | X | X | X | | X | | | L. MCA |
| | | | | | | | | | | | | | | | ischemia, |
| | | | | | | | | | | | | | | | possible L. |
| | | | | | | | | | | | | | | | insula |
| | | | | | | | | | | | | | | | infarction |
| 6 | M | 45 | 0 | 36 | 6 | 1 | −5 | | | X | X | | | | No acute |
| | | | | | | | | | | | | | | | hemorrhage |
| | | | | | | | | | | | | | | | or territorial |
| | | | | | | | | | | | | | | | infarct |
| 7 | F | 86 | 0 | 69 | 4 | 4 | 0 | X | X | | | | X | | R. Thalamic |
| | | | | | | | | | | | | | | | ICH with IVH |
| | | | | | | | | | | | | | | | extension. |
| | | | | | | | | | | | | | | | ACA |
| | | | | | | | | | | | | | | | aneurysm |
| 8 | M | 52 | 0 | 38 | 11 | 9 | −2 | X | X | | X | | X | | L. Thalamic |
| | | | | | | | | | | | | | | | hemorrhage |

NIHSS, National Institutes of Health Stroke Scale; MSU, mobile stroke unit; mRS, modified Rankin Scale; ED, emergency department; CT, computed tomography; CTA, CT angiography; R, right; L, left; M1, M1 segment of middle cerebral artery; M2, M2 segment of middle cerebral artery; MCA, middle cerebral artery; ICH, intracerebral hemorrhage; ACA, anterior communicating artery skilled nursing facility.
† Derived from clinical radiology reports.
‡ Acute change in NIHSS score from initial evaluation on MSU to first subsequent evaluation in ED. [symbol]
indicates data missing or illegible when filed

TABLE 3
Blood-based neuroinflammatory factor levels of patients with confirmed stroke treated on mobile stroke unit

| | | | | Clinical blood samples - ED | | | | |
|---|---|---|---|---|---|---|---|---|
| | | | | | UCH clinical lab | | | |
| | | | | Time | testing | | | |
| | | | | from | | Abs | Abs | Abs |
| | | | | stroke | WBC | Neutrophils | Lymphocytes | Monocytes |
| Patient characteristics | | | | symptom | (10⁹/L) | (10⁹/L) | (10⁹/L) | (10⁹/L) |
| Patient | | | Stroke | onset | Ref: | Ref: | Ref: | Ref: |
| no. | Sex | Age | Diagnosis | (mins) | 4-11.1 | 1.8-6.6 | 1.0-4.8 | 0.2-0.9 |
| 1 | M | 85 | AIS | 116 | 4.6 | 3.0 | 3.0 | 0.4 |
| 2 | F | 36 | AIS | N/A | N/A | N/A | N/A | N/A |
| 3 | F | 59 | AIS | 242 | 12.5↑ | 9.7↑ | 2.0 | 0.6 |
| 4 | M | 76 | AIS | 193 | 11.2↑ | 9.1↑ | 1.0 | 1.0↑ |
| 7 | F | 86 | ICH | 107 | 12.4↑ | 11.1↑ | 0.7↓ | 0.4 |
| 8 | M | 52 | ICH | 84 | 8.9 | 5.9 | 2.4 | 0.5 |
| 5 | M | 78 | AIS | 151 | 10.6 | N/A | N/A | N/A |
| 6 | M | 45 | AIS | 87 | 7.3 | 3.7 | 2.4 | 0.7 |

| | Ultra-early biomarker blood samples - MSU | | | | | | | | | | |
|---|---|---|---|---|---|---|---|---|---|---|---|
| | | | SIMOA | | | | | | | | |
| | Time | ECL | NfL | UCH-L1 | | Extracellular vesicles isolated by HSP pull down | | | | | |
| | from | IL-6 | (pg/mL) | (pg/mL) | | CRP | MMP-9 | CXCL4 | PECAM1 | IL-6 | OPN |
| | stroke | (pg/mL) | Control: | Control: | GFAP | (pixel | (pixel | (pixel | (pixel | (pixel | (pixel |
| | symptom | Ref: | 4.09 | 4.09 | (pg/mL) | density) | density) | density) | density) | density) | density) |
| Patient | onset | 1.89 | (1.34- | (1.34- | Control: | Control: | Control: | Control: | Control: | Control: | Control: |
| no. | (mins) | (0.3-5.0) | [symbol] | [symbol] | 80 (56-133) | 8959 | 1765 | 702 | 1804 | 502 | 2451 |
| 1 | 83 | 9.2↑ | 40.8↑ | 52.6↑ | 156↑ | 26823↑ | 4265↑ | 14781↑ | 5280↑ | 510 | 12432↑ |
| 2 | 48 | 5.3↑ | 1.9 | 28.0↑ | 53↑ | 20729↑ | 1542 | 13648↑ | 5309↑ | 653↑ | 7507↑ |
| 3 | 102 | 16.8↑ | 130.8↑ | 44.2↑ | 234↑ | 10758↑ | 4708↑ | 6771↑ | 4756↑ | 1047↑ | 3409↑ |
| 4 | 133 | 36.8↑ | 21.3↑ | 164.2↑ | 282↑ | 12875↑ | 8134↑ | 9734↑ | 2739↑ | 1047↑ | 4639↑ |
| 7 | 69 | 13.1↑ | 31.9↑ | 86.0↑ | 1527↑ | N/A | N/A | N/A | N/A | N/A | N/A |
| 8 | 38 | 1.8 | 7.72↑ | 20.5↑ | 71↑ | 11774↑ | 5863↑ | 7503↑ | 4486↑ | 1859↑ | 5557↑ |
| 5 | 40 | 3.5↑ | N/A | N/A | N/A | N/A | N/A | N/A | N/A | N/A | N/A |
| 6 | 36 | 3.8↑ | N/A | N/A | N/A | N/A | N/A | N/A | N/A | N/A | N/A |

ED, emergency department; MSU, mobile stroke unit; UCH, University of Colorado Hospital; ECL, electrochemiluminescence immunoassay; SIMOA, single molecule array; HSP, heat-shock protein; AIS, acute ischemic stroke; ICH, intracerebral hemorrhage; WBC, white blood count; IL-6, interleukin 6; NfL, neurofilament light chain; UCH-L1, ubiquitin C-terminal hydrolase L1; GFAP, glial fibrillary astrocytic protein; CRP, C-reactive protein; MMP-9, matrix metalloproteinase 9; CXCL4, C-X-C motif ligand 4; PECAM-1, platelet endothelial cell adhesion molecule-1; OPN, osteopontin.
↑—Above reference level or control.
↓—Below reference level or control. [symbol]
indicates data missing or illegible when filed

TABLE 4
Plasma neuroinflammatory factors in MSU stroke patients and stroke mimics stroke

| Blood-based | | | All Patients | | Confirmed Stroke | | Stroke Mimic^{**} | |
|---|---|---|---|---|---|---|---|---|
| markers^{*} | Normal Range^{†} | | (N = 15) | | (N = 8) | | (N = 7) | |
| Plasma pro- | | | | | | | | |
| neuroinflammatory | | | | | | | | |
| factors | | | | | | | | |
| Minutes from | | | 69 | (35-835) | 46 | (36-133) | 405 | (35-835) |
| stroke symptom | | | | | | | | |
| onset | | | | | | | | |
| IL-17A (pg/mL) | 0.091 | (0.030-0.90) | 0.51 | (0.12-1.42) | 0.36 | (0.12-1.42) | 0.56 | (0.21-1.30) |
| Normal | | | 15 | (100) | 8 | (100) | 7 | (100) |
| Elevated | | | 0 | (0) | 0 | (0) | 0 | (0) |
| IL-1β (pg/mL) | 0.070 | (ND-5.90) | 0.14 | (0.05-4.35) | 0.11 | (0.05-0.29) | 0.14 | (0.09-4.35) |
| Normal | | | 14 | (94) | 8 | (100) | 6 | (86) |
| Elevated | | | 1 | (6) | 0 | (0) | 1 | (14) |
| IL-6 (pg/mL) | 1.60 | (0.330-5.80) | 7.31 | (1.76-282.98) | 7.27 | (1.76-36.76) | 7.31 | (1.78-282.98) |
| Normal^{‡} | | | 2 | (13) | 1 | (12) | 1 | (14) |
| Elevated^{‡} | | | 13 | (87) | 7 | (88) | 6 | (86) |
| TNFα (pg/mL) | 0.170 | (0.038-1.70) | 0.53 | (0.34-5.70) | 0.47 | (0.34-0.74) | 0.55 | (0.41-5.70) |
| Normal | | | 14 | (94) | 8 | (100) | 6 | (86) |
| Elevated | | | 1 | (6) | 0 | (0) | 1 | (14) |

Data are N (%), median (range).
MSU, mobile stroke unit; IL-17A, interleukin 17A; IL-1β, interleukin 1β; IL-6, interleukin 6; TNFα, tumor necrosis factor alpha; ND, not detected.
^{*}Plasma neuroinflammatory factors measured by electrochemiluminescence assay (ECL).
^{**}Includes one patient with suspected TIA, and one patient with a possible ischemic stroke aborted by tPA.
^{†}Mesoscale Discovery S-PLEX Proinflammatory Panel 1 human insert. www.mesoscale.com/~/media/files/product%20inserts/s-plex%20proinflammatory%20panel%201%20human%20insert.pdf. Accessed Aug. 6, 2022.
^{‡}Normal and elevated IL-6 based on reference range: median 1.89 pg/mL, upper 95^{th} percentile 4.45 pg/mL. Todd et al.

TABLE 5
Comparison of stroke-onset and 24-hour plasma neuroinflammatory factors in stroke patients with ECL

| | | | Ultra-early | | 24-hour | | | |
|---|---|---|---|---|---|---|---|---|
| Blood-based | | | (on MSU) | | (acute hospital care) | | Δ | |
| markers | Normal Range^{†} | | N = 8 | | N = 6 | | (MSU to 24 h) | |
| Minutes from stroke | | | 46 | (36-133) | 1435 | (1215-1596) | | |
| symptom onset | | | | | | | | |
| Pro-inflammatory | | | | | | | | |
| cytokines | | | | | | | | |
| IL-17A (pg/mL) | 0.091 | (0.030-0.90) | 0.36 | (0.12-1.42) | 0.35 | (0.19-2.42) | +0.07 | (−0.19 to ++1.33) |
| Normal | | | 8 | (100) | 5 | (83) | | |
| Elevated | | | 0 | (0) | 1 | (17) | | |
| IL-1β (pg/mL) | 0.070 | (ND-5.90) | 0.11 | (0.05-0.29) | 0.21 | (0.05-0.56) | +0.11 | (−0.12 to +0.44) |
| Normal | | | 8 | (100) | 6 | (100) | | |
| Elevated | | | 0 | (0) | 0 | (0) | | |
| IL-6 (pg/mL) | 1.60 | (0.330-5.80) | 7.27 | (1.76-36.76) | 39.74 | (5.64-94.05) | +12.96 | (+3.88 to +84.79) |
| Normal^{‡} | | | 1 | (12) | 0 | (0) | | |
| Elevated^{‡} | | | 7 | (88) | 6 | (100) | | |
| TNFα (pg/mL) | 0.160 | (0.110-5.50) | 0.47 | (0.34-0.74) | 0.76 | (0.35-1.03) | +0.21 | (0.0 to +0.39) |
| Normal | | | 8 | (100) | 6 | (100) | | |
| Elevated | | | 0 | (0) | 0 | (0) | | |
| IFN-γ (pg/mL) | 0.170 | (0.038-1.70) | 0.51 | (0.10-41.15) | 0.18 | (0.10-56.10) | −0.05 | (−0.83 to +14.95) |
| Normal | | | 8 | (100) | 6 | (100) | | |
| Elevated | | | 0 | (0) | 0 | (0) | | |

Data are N (%), median (range).
MSU, mobile stroke unit, ECL, electrochemiluminescence assay; IL-17A, interleukin 17A; IL-1β, interleukin 1β; IL-6, interleukin 6; TNFα, tumor necrosis factor alpha; IFN-γ, interferon gamma; ND, none detected.
^{†}Mesoscale Discovery S-PLEX Proinflammatory Panel 1 human insert. www.mesoscale.com/~/media/files/product%20inserts/s-plex%20proinflammatory%20panel%201%20human%20insert.pdf. Accessed Aug. 6, 2022.
^{‡}Normal and elevated IL-6 based on reference range: median 1.89 pg/mL, upper 95^{th} percentile 4.45 pg/mL. Todd J., Simpson P., Estis J., Torres V., Wub A. H. Reference range and short- and long-term biological variation of interleukin (IL)-6, IL-17A and tissue necrosis factor-alpha using high sensitivity assays. Cytokine. December 2013; 64(3): 660-665.

TABLE 6
Comparison of plasma neuroinflammatory factors and stroke proteins in stroke patients and controls with ELISA

| Blood-based | | | Ultra-early | | 24-hr | | Δ | |
|---|---|---|---|---|---|---|---|---|
| markers | Healthy Controls | | (MSU) | | (acute hospital care) | | (MSU to 24 h) | |
| Plasma pro- | N = 5 | | N = 7 | | N = 5 | | N = 5 | |
| neuroinflammatory | | | | | | | | |
| factors | | | | | | | | |
| Minutes from stroke | | | 48 | (38-133) | 1,473 | (1,215-1,596) | | |
| symptom onset | | | | | | | | |
| IL-6 (pg/mL) | 2.30 | (0.93-6.22) | 7.36 | (2.03-41.05) | 41.03 | (4.31-54.75) | +10.97 | (+2.28 to +47.39) |
| Normal^{†} | 3 | (60) | 2 | (29) | 1 | (20) | | |
| Elevated^{†} | 2 | (40) | 5 | (71) | 4 | (80) | | |
| Δ during first 24 h | | | | | | | | |
| Increase | | | | | | | 5 | (100) |
| Decrease | | | | | | | 0 | (0) |
| Plasma stroke | | | | | | | | |
| proteins | | | | | | | | |
| NSE (pg/mL) | N/A | | 9,244 | (6,610-22,067) | 4,552 | (2,709-6,728) | −4,953 | (−1,964 to −18,014) |
| Normal | | | N/A | | N/A | | | |
| Elevated | | | N/A | | N/A | | | |
| NfL (pg/mL) | 24.66 | (9.46-26.37) | 31.94 | (1.94-130.82) | 17.82 | (2.36-112.52) | +0.42 | (−18.30 to +13.17) |
| Normal^{*} | | | 3 | (43) | 3 | (60) | | |
| Elevated^{*} | | | 4 | (57) | 2 | (40) | | |
| UCH-L1 (pg/mL) | 18.89 | (11.88-61.61) | 44.19 | (20.54-85.98) | 25.84 | (10.99-164.19) | −8.39 | (−17.06 to +120.00) |
| Normal^{*} | | | 0 | (0) | 2 | (40) | | |
| Elevated^{*} | | | 7 | (100) | 3 | (60) | | |
| GFAP (pg/mL) | 187.87 | (91.19-359.53) | 184.90 | (52.77-1,526.74) | 312.74 | (53.03-3,537.32) | +221.90 | (+0.26 to +3,303.05) |
| Normal^{*} | | | 4 | (57) | 1 | (20) | | |
| Elevated^{*} | | | 3 | (43) | 4 | (80) | | |

Data are N (%), median (range).
ELISA, enzyme-linked immunoassay; MSU, mobile stroke unit; IL-6, interleukin 6; NSE, neuron specific enolase; NfL, neurofilament light chain; UCH-L1, ubiquitin C-terminal hydrolase L1; GFAP, glial fibrillary acidic protein.
^{†}Normal and elevated IL-6 based on reference range: median 1.89 pg/mL, upper 95^{th} percentile 4.45 pg/mL. Todd et al.
^{*}Compared with median plasma concentration in healthy controls.

TABLE 7
Comparison of plasma neuroinflammatory factors and stroke proteins in stroke patients and controls with SIMOA

| Blood-based | | | Ultra-early | | 24-hour | | Δ | |
|---|---|---|---|---|---|---|---|---|
| markers | Healthy Controls | | (MSU) | | (acute hospital care) | | (MSU to 24 h) | |
| Plasma proteins | N = 21 | | N = 6 | | N = 5 | | N = 5 | |
| Time from | | | 76 | (38-133) | 1,473 | (1,215-1,596) | | |
| stroke symptom | | | | | | | | |
| onset (mins) | | | | | | | | |
| NfL (pg/mL) | 4.09 | (1.34-8.84) | 26.63 | (1.94-130.82) | 17.82 | (2.36-112.52) | +0.42 | (−18.30 to +13.17) |
| Normal^{†} | | | 1 | (17) | 1 | (20) | | |
| Elevated^{‡} | | | 5 | (83) | 4 | (80) | | |
| Δ first 24 h | | | | | | | 3 | (60) |
| Increase | | | | | | | 2 | (40) |
| Decrease | | | | | | | | |
| UCH-L1 | 17.38 | (2.98-32.91) | 41.27 | (20.54-85.98) | 25.84 | (10.99-164.19) | −8.39 | (−17.06 to +120.00) |
| (pg/mL) | | | | | | | | |
| Normal | | | 0 | (0) | 2 | (40) | | |
| Elevated | | | 6 | (100) | 3 | (60) | | |
| Δ first 24 h | | | | | | | | |
| Increase | | | | | | | 2 | (40) |
| Decrease | | | | | | | 3 | (60) |
| GFAP (pg/mL) | 80.37 | (56.43-132.86) | 195.22 | (52.77-1,526.74) | 312.70 | (53.00-3,537.30) | +221.86 | (+0.23 to +3,303.03) |
| Normal | | | 2 | (33) | 1 | (20) | | |
| Elevated | | | 4 | (67) | 4 | (80) | | |
| Δ first 24 h | | | | | | | | |
| Increase | | | | | | | 5 | (100) |
| Decrease | | | | | | | 0 | (0) |

Data are N (%), median (range).
SIMOA, single molecule array; MSU, mobile stroke unit; NfL, neurofilament light chain; UCH-L1, ubiquitin C-terminal hydrolase L1; GFAP, glial fibrillary acidic protein.
^{†}Compared with median plasma concentration in healthy controls.

### Extracellular Vesicle-Derived—Neuroinflammatory Factor Levels

Among inflammatory factors with known association with stroke, CRP (C-reactive protein), MMP-9, CXCL4, PECAM1 (platelet and endothelial cell adhesion molecule 1), IL-6, and OPN (osteopontin) were higher for confirmed stroke patients in the minutes after symptom onset, than for healthy controls, both in terms of medians, and in individual patient values in 97% o of cases (Table S7; Figures S4 and S5; Additional results, see Supplemental Material, Figure S6, Tables S8 through S11).

TABLE 8
Extracellular vesicle-derived neuroinflammatory factors in confirmed stroke patients

| | Healthy | Ultra-early | | 24-hour | | Δ | |
|---|---|---|---|---|---|---|---|
| | Controls | (MSU) | | (acute hospital care) | | (MSU to 24 h) | |
| EV-based | (Pooled | N = 5 | | N = 5 | | N = 5 | |
| neuroinflammatory | HC) | | | | | | |
| factors | | | | | | | |
| Time from stroke | | 83 | (38-133) | 1,473 | (1,215-1,596) | | |
| symptom onset | | | | | | | |
| (mins) | | | | | | | |
| CRP (pixel density) | 8,959 | 12,875 | (10,758-26,823) | 16,473 | (12,042-33,008) | +3,598 | (+268 to +6,185) |
| Normal^{†} | | 0 | (0) | 0 | (0) | | |
| Elevated^{†} | | 5 | (100) | 5 | (100) | | |
| Δ first 24 h | | | | | | 5 | (100) |
| Increase | | | | | | 0 | (0) |
| Decrease | | | | | | | |
| MMP-9 (pixel | 1,765 | 4,708 | (1,542-8,134) | 6,846 | (1,087-16,473) | +983 | (−3,621 to +8,339) |
| density) | | | | | | | |
| Normal | | 0 | (0) | 1 | (20) | | |
| Elevated | | 5 | (100) | 4 | (80) | | |
| Δ first 24 h | | | | | | | |
| Increase | | | | | | 2 | (40) |
| Decrease | | | | | | 3 | (60) |
| CXCL4 (pixel | 702 | 9,734 | (6,771-14,781) | 11,684 | (5,450-14,932) | +151 | (−1,321 to +1,950) |
| density) | | | | | | | |
| Normal | | 0 | (0) | 0 | (0) | | |
| Elevated | | 5 | (100) | 5 | (100) | | |
| Δ first 24 h | | | | | | | |
| Increase | | | | | | 3 | (60) |
| Decrease | | | | | | 2 | (40) |
| PECAM1 (pixel | 1,804 | 4,756 | (2,739-5,309) | 3,582 | (2,171-5,948) | −568 | (−2,206 to +1,192) |
| density) | | | | | | | |
| Normal | | 0 | (0) | 0 | (0) | | |
| Elevated | | 5 | (100) | 5 | (100) | | |
| Δ first 24 h | | | | | | | |
| Increase | | | | | | 2 | (40) |
| Decrease | | | | | | 3 | (60) |
| IL-6 (pixel density) | 510 | 1,047 | (510-1,859) | 942 | (570-2,973) | +60 | (−105 to +1,926) |
| Normal | | 1 | (20) | 0 | (0) | | |
| Elevated | | 4 | (80) | 5 | (100) | | |
| Δ first 24 h | | | | | | | |
| Increase | | | | | | 3 | (60) |
| Decrease | | | | | | 2 | (40) |
| OPN (pixel density) | 2,451 | 5,557 | (3,409-12,432) | 6,758 | (3,874-12,575) | +987 | (+143 to +1.646) |
| Normal | | 0 | (0) | 0 | (0) | | |
| Elevated | | 5 | (100) | 5 | (100) | | |
| Δ first 24 h | | | | | | | |
| Increase | | | | | | 5 | (100) |
| Decrease | | | | | | 0 | (0) |

Data are N (%), median (range).
MSU, mobile stroke unit; EV, extracellular vesicle; CRP, C-reactive protein; MMP-9, matrix metalloproteinase-9; CXCL4, chemokine (C-X-C motif) ligand 4; PECAM1, platelet and endothelial cell adhesion molecule 1; IL-6, interleukin-6; OPN, osteopontin.

TABLE 9
Acute leukocyte levels in MSU patients after stroke onset

| | | All | | Confirmed | | Stroke | |
|---|---|---|---|---|---|---|---|
| Clinical leukocyte | Normal | Patients | | Stroke | | Mimic* | |
| values (CBC) | Range^{†} | (N = 15) | | (N = 8) | | (N = 7) | |
| Minutes from stroke | | 133 | (79-865) | 116 | (84-242) | 452 | (79-865) |
| symptom onset | | | | | | | |
| WBC (10⁹/L) | 4-11.1 | 9.3 | (4.6-21.1) | 10.6 | (4.6-12.5) | 8.6 | (6.5-21.1) |
| Normal | | 10/14 | (71) | 4/7 | (57) | 6 | (86) |
| Elevated | | 4/14 | (29) | 3/7 | (43) | 1 | (14) |
| Neutrophils (10⁹/L) | 1.8-6.6 | 5.8 | (3-17.6) | 7.5 | (3-11.1) | 5.4 | (4.7-17.6) |
| Normal | | 8/13 | (61) | 3/6 | (50) | 5 | (71) |
| Elevated | | 5/13 | (38) | 3/6 | (50) | 2 | (29) |
| Lymphocytes | 1.0-4.8 | 1.6 | (0.7-3.0) | 1.5 | (0.7-2.4) | 1.6 | (1.0-3.0) |
| (10⁹/L) | | | | | | | |
| Normal | | 13/13 | (100) | 6/6 | (100) | 7 | (100) |
| Elevated | | 0 | (0) | 0 | (0) | 0 | (0) |
| Monocytes (10⁹/L) | 0.2-0.9 | 0.7 | (0.4-1.6) | 0.5 | (0.4-1.0) | 0.8 | (0.4-1.6) |
| Normal | | 11 | (73) | 5/6 | (83) | 6 | (86) |
| Elevated | | 2 | (13) | 1/6 | (17) | 1 | (14) |
| Eosinophils (10⁹/L) | 0.0-0.4 | 0.0 | (0.0-0.3) | 0.5 | (0.0-0.3) | 0.0 | (0.0-0.3) |
| Normal | | 13/13 | (100) | 6/6 | (100) | 7 | (100) |
| Elevated | | 0 | (0) | 0 | (0) | 0 | (0) |

Data are N (%), median (range).
CBC, complete blood count; MSU, mobile stroke unit.
*Includes one patient with suspected TIA, and one patient with a possible ischemic stroke aborted by tPA.
^{†}University of Colorado Hospital clinical reference ranges.

TABLE 10
Summary table of normal reference ranges for study biomarker comparisons

| Biomarker | | | | | | N |
|---|---|---|---|---|---|---|
| test utilized | | | Normal | | | evaluated |
| for study | | | reference values | | | for normal |
| subject plasma | Biomarker | Units | [median, (range)] | | Normal range source | range |
| SIMOA | NfL | pg/mL | 4.09 | (1.34-8.84) | Study healthy adult | 21 |
| | UCH-L1 | | 17.38 | (2.98-32.91) | controls | |
| | GFAP | | 80.37 | (56.43-132.86) | | |
| ELISA | NfL | pg/mL | 24.66 | (9.46-26.37) | Study healthy adult | 5 |
| | UCH-L1 | | 18.89 | (11.88-61.61) | controls | |
| | GFAP | | 187.87 | (91.19-359.53) | | |
| ECL | IL-17A | pg/mL | 0.091 | (0.030-0.90) | MSD S-Plex reference¹ | 17 |
| | IL-1β | | 0.070 | (ND-5.90) | | |
| | TNFα | | 0.160 | (0.110-5.50) | | |
| | IFN-γ | | 0.170 | (0.038-1.70) | | |
| | IL-6 | | 1.60 | (0.330-5.80) | | |
| | | | 1.89 | (4.45)^{†} | Todd et al, Cytokine² | 125 |

| | | | ≤1.8 | Mayo Clinic | N/A |
|---|---|---|---|---|---|
| | | | | Laboratories³ | |
| EV* | CRP | pixel | 8,959 | Pooled sample healthy | Pooled |
| | MMP-9 | density | 1,765 | adults^{**} | |
| | CXCL4 | | 702 | | |
| | PECAM1 | | 1,804 | | |
| | IL-6 | | 510 | | |
| | OPN | | 2,451 | | |

SIMOA, single molecule array; ELISA, enzyme-linked immunoassay; ECL, electrochemiluminescence assay; EV, extracellular vesicle; NfL, neurofilament light chain; UCH-L1, ubiquitin C-terminal hydrolase L1; GFAP, glial fibrillary acidic protein; IL-17A, interleukin 17A; IL-1β, interleukin 1β; TNFα, tumor necrosis factor alpha; IFN-γ, interferon gamma; IL-6, interleukin 6; CRP, C-reactive protein; MMP-9, matrix metalloproteinase-9; CXCL4, chemokine (C-X-C motif) ligand 4; PECAM1, platelet and endothelial cell adhesion molecule 1; OPN, osteopontin.
*Evs isolated from study subject plasma. Biomarkers quantified with membrane-based sandwich immunoassay (Proteome Profiler Human XL Cytokine Array).
^{**}Pooled plasma-derived serum sample, Innovative Research, MI, USA, ISERAB-100 mL.
^{†}Normal reference range reported as mean (upper 95^{th} percentile).
¹Mesoscale Discovery S-PLEX Proinflammatory Panel 1 human insert. www.mesoscale.com/~/media/files/product%20inserts/s-plex%20proinflammatory%20panel%201%20human%20insert.pdf. Accessed Aug. 6, 2022.
²Todd J., Simpson P., Estis J., Torres V., Wub A. H. Reference range and short- and long-term biological variation of interleukin (IL)-6, IL-17A and tissue necrosis factor-alpha using high sensitivity assays. Cytokine. December 2013; 64(3): 660-665.
³Mayo Clinic Laboratories Plasma Interleukin-6 Clinical Information. www.mayocliniclabs.com/test-catalog/Overview/63020#Clinical-and-Interpretive. Accessed Jun. 8, 2022.

TABLE 11
Association of blood sample processing times with biomarker concentrations

| Blood sample handling times confirmed stroke patients | | |
|---|---|---|
| Time from MSU | Time from 4° C. | Time from MSU |
| blood draw to 4° C. | to −80° C. | blood draw to −80° C. |

Time in
transition
(minutes)

| All stroke | 23 (12-37) | | 1,501 (339-4,058) | | 1,521 (351-4,093) | |
|---|---|---|---|---|---|---|
| patients (N = 8) | | | | | | |
| ELISA (N = 7) | 21 (12-37) | | 1,471 (339-4,058) | | 1,490 (351-4,093) | |
| SIMOA (N = 6) | 20 (12-37) | | 1,388 (339-1,553) | | 1,406 (351-1,590) | |
| ECL (N = 8) | 23 (12-37) | | 1,501 (339-4,058) | | 1,521 (351-4,093) | |
| EV (N = 5) | 21 (16-37) | | 1,471 (1,113-1,553) | | 1,490 (1,148-1,590) | |
| Immunoassay | | | | | | |
| and | Spearman's | | Spearman's | | Spearman's | |
| Inflammatory | Correlation | | Correlation | | Correlation | |
| Factor | coefficient | P value | coefficient | P value | coefficient | P value |
| ELISA (N = 7) | | | | | | |
| IL-6 | −0.108 | 0.818 | −0.500 | 0.253 | −0.500 | 0.253 |
| NSE | 0.314 | 0.544 | 0.371 | 0.468 | 0.371 | 0.468 |
| NfL | −0.270 | 0.558 | 0.143 | 0.760 | 0.143 | 0.760 |
| UCH-L1 | −0.450 | 0.310 | −0.214 | 0.645 | −0.214 | 0.645 |
| GFAP | −0.396 | 0.379 | −0.607 | 0.148 | −0.607 | 0.148 |
| SIMOA (N = 6) | | | | | | |
| NfL | −0.486 | 0.329 | −0.257 | 0.623 | −0.257 | 0.623 |
| UCH-L1 | −0.657 | 0.156 | −0.600 | 0.208 | −0.600 | 0.208 |
| GFAP | −0.468 | 0.329 | −0.829 | 0.042 | −0.829 | 0.042 |
| ECL (N = 8) | | | | | | |
| IL-17A | −0.407 | 0.317 | −0.595 | 0.120 | −0.524 | 0.310 |
| IL-1β | 0.132 | 0.756 | −0.119 | 0.779 | −0.095 | 0.823 |
| IL-6 | −0.072 | 0.866 | −0.595 | 0.120 | −0.571 | 0.139 |
| TNFα | 0.347 | 0.399 | 0.024 | 0.955 | 0.095 | 0.823 |
| IFN-γ | 0.275 | 0.509 | −0.357 | 0.385 | −0.238 | 0.570 |
| Extracellular | | | | | | |
| Vesicles (N = 5) | | | | | | |
| CRP | −0.100 | 0.873 | −0.200 | 0.747 | −0.200 | 0.747 |
| MMP-9 | −0.100 | 0.873 | −0.700 | 0.188 | −0.700 | 0.188 |
| CXCL4 | −0.100 | 0.873 | −0.200 | 0.747 | −0.200 | 0.747 |
| PECAM1 | −0.100 | 0.873 | 0.700 | 0.188 | 0.700 | 0.188 |
| IL-6 | 0.051 | 0.935 | −0.051 | 0.935 | −0.051 | 0.935 |
| OPN | −0.300 | 0.624 | 0.000 | 1.000 | 0.000 | 1.000 |

Data are median (range).
ELISA, enzyme-linked immunosorbent assay; SIMOA, single molecule array; ECL, electrochemiluminescence immunoassay; EV, extracellular vesicle; MSU, mobile stroke unit; IL-6, interleukin 6; NSE, neuron-specific enolase; NfL, neurofilament light chain; UCH-L1, ubiquitin C-terminal hydrolase L1; GFAP, glial fibrillary acidic protein; IL-17A, interleukin 17A; IL-1β, interleukin 1β; CRP, C-reactive protein; MMP-9, matrix metalloproteinase 9; CXCL4, C-X-C motif ligand 4; PECAM-1, platelet endothelial cell adhesion molecule-1; OPN, osteopontin.

TABLE 12
Acute hospitalization outcomes in patients treated on mobile stroke unit

| | | | Confirmed | | Stroke | |
|---|---|---|---|---|---|---|
| | All Patients | | Stroke | | Mimic* | |
| Outcome Characteristics | (N = 15) | | (N = 8) | | (N = 7) | |
| Clinical and functional disability | | | | | | |
| status | | | | | | |
| NIHSS score at hospital discharge | 4 | (0-6)^{†} | 4.5 | (1-9) | 0 | (0-0)^{‡} |
| 0-4. Minor | 7 | (64) | 4 | (50) | 3 | (100) |
| 5-15. Moderate | 3 | (27) | 3 | (37) | 0 | (0) |
| 16-20. Moderate to severe | 1 | (9) | 1 | (12) | 0 | (0) |
| 21-42. Severe | 0 | (0) | 0 | (0) | 0 | (0) |
| Δ NIHSS score (MSU to hospital | −6 | (−1 to −7) | −5.5 | (−0.25 to −9.25) | −6 | (−1 to −7) |
| discharge) | | | | | | |
| Improved | 9 | (82) | 6 | (75) | 3 | (100) |
| Unchanged | 1 | (9) | 1 | (12) | 0 | (0) |
| Worsened | 1 | (9) | 1 | (12) | 0 | (0) |
| Modified Rankin Scale score at | | | | | | |
| hospital discharge | | | | | | |
| 0. No symptoms | 4 | (27) | 2 | (25) | 2 | (29) |
| 1. No significant disability | 1 | (7) | 0 | (0) | 1 | (14) |
| 2. Slight disability | 2 | (13) | 2 | (25) | 0 | (0) |
| 3. Moderate disability | 2 | (13) | 2 | (25) | 0 | (0) |
| 4. Moderately severe disability | 5 | (33) | 1 | (12) | 4 | (57) |
| 5. Severe Disability | 1 | (7) | 1 | (12) | 0 | (0) |
| 6. Death | 0 | (0) | 0 | (0) | 0 | (0) |
| Discharge Disposition | | | | | | |
| Home | 8 | (53) | 3 | (37) | 5 | (71) |
| Inpatient Stroke rehabilitation | 3 | (20) | 3 | (37) | 0 | (0) |
| Skilled nursing facility | 2 | (13) | 1 | (12) | 1 | (14) |
| Hospice care | 2 | (13) | 1 | (12) | 1 | (14) |

Data are N (%), median (IQR).
NIHSS, National Institutes of Health Stroke Scale; MSU, mobile stroke unit.
*Includes one patient with suspected TIA, and one patient with a possible ischemic stroke
aborted by tPA.
^{†}Discharge NIHSS score available for 11 of 15 patients treated on mobile stroke unit.
^{‡}Discharge NIHSS score available for 3 of 7 stroke mimic patients.

### Supplemental Results

### Patient Population

Of study patients treated on the MSU and diagnosed as stroke, 2 were eventually diagnosed as likely transient ischemic attack (TIA) or stroke averted by thrombolysis. Of patients with confirmed stroke 2 (25%) were being treated with antiplatelet therapy at the time of stroke onset. The remaining 5 patients were stroke mimics, including: seizures, COPD exacerbation, pulmonary embolism and alcohol withdrawal Among confirmed strokes, 6 (75%) were acute ischemic stroke (AIS) involving middle cerebral artery (MCA) territories, and 2 (25%) were thalamic intracerebral hemorrhage (ICH). Median initial NIHSS score was 11 (range 4-19), and 3 (37%) patients improved before arrival at the emergency department (ED), while 4 (50%) worsened. Six (75%) of confirmed stroke patients received IV tPA, and this was administered on the MSU in the field to 4 (67%), and after arrival at the ED in 2 (33%) patients. Two (33%) AIS patients underwent mechanical thrombectomy. Median length of hospital stay for confirmed stroke patients was 4 (range 2-7) days (Table S1). Of all healthy controls, 14 (54%) were female, and median age was 21 (range 18-86) years. In controls used in SiMoA analyses, 11 (52%) were female, and median age was 21 (range 18-26) years. Among controls in the ELISA analyses, 3 (60%) were female and the median age was 70 (range 65-86) years.

### Blood Sampling

Peripheral venous blood was obtained at the patients' homes for measurement of neuroinflammatory markers at a median of 58 (range 36-133) minutes after stroke symptom onset for all 8 confirmed stroke cases. IL-6 is known to have diurnal variation, with a morning trough of approximately 0.5 pg/mL between 6 a.m. and 12 noon.19 In our cohort, no association was found between the time of day blood was drawn and IL-6 levels. For confirmed stroke patients, median time between the initial blood draw on the MSU and refrigeration at 4° C. was 23 (range 12-37) minutes, and median time from the MSU blood draw to freezing at −80° C. was 1,501 (range 339-4,058) minutes. No correlation was found between blood processing times and inflammatory factor levels in any of the blood or extracellular vesicle assays used in the study (Table 11).

### Acute Outcomes

At hospital discharge, median NIHSS score for confirmed stroke patients was 4 (range 1-9). NIHSS severity was minor in 4 (50%) patients, moderate in 3 (37%) and moderate to severe in 1 (12%). Six (75%) patients improved from initial evaluation, 1 (12%) was unchanged and 1 (12%) worsened. Four (50%) patients had favorable functional disability (Modified Rankin Scale score≤2), 3 (37%) were discharged to home, 3 (37%) to inpatient rehabilitation, 1 (12%) to a skilled nursing facility and 1 (12%) to hospice (Table 12).

### Plasma Neuroinflammatory Factor Levels

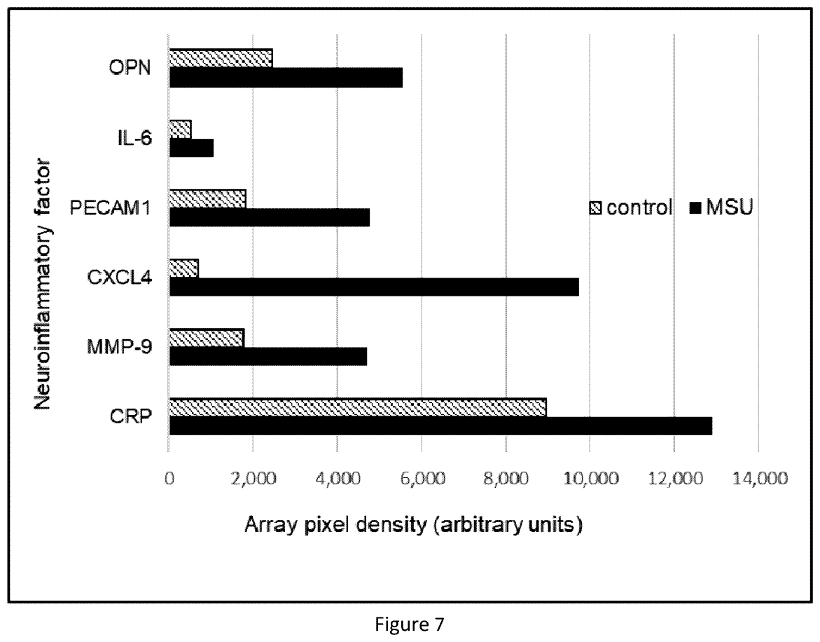

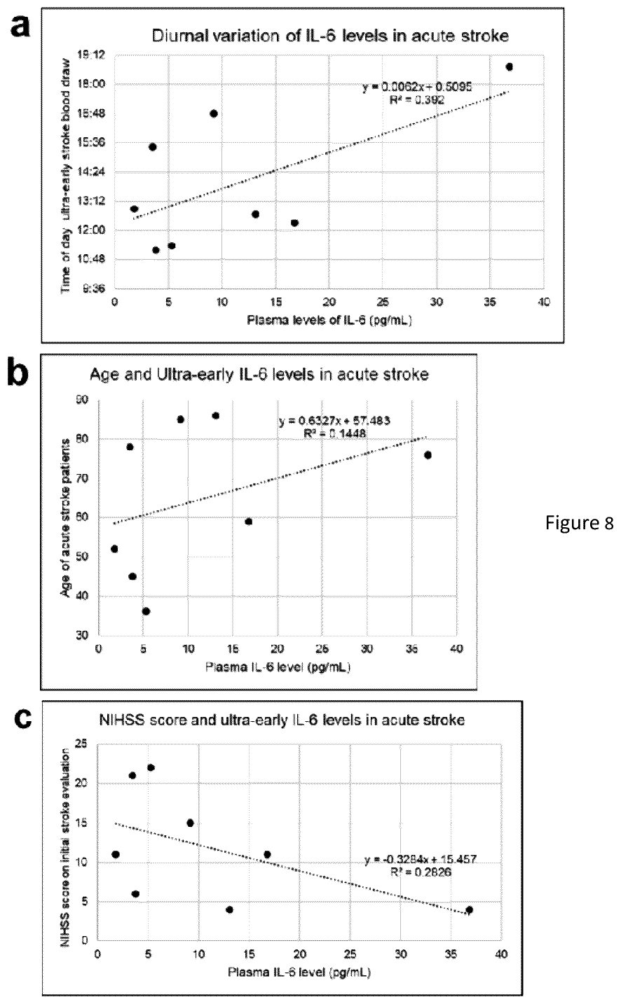

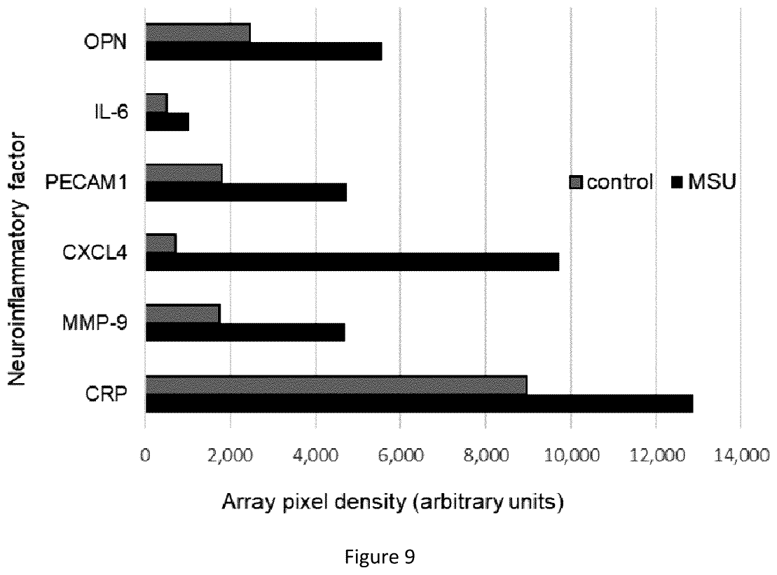

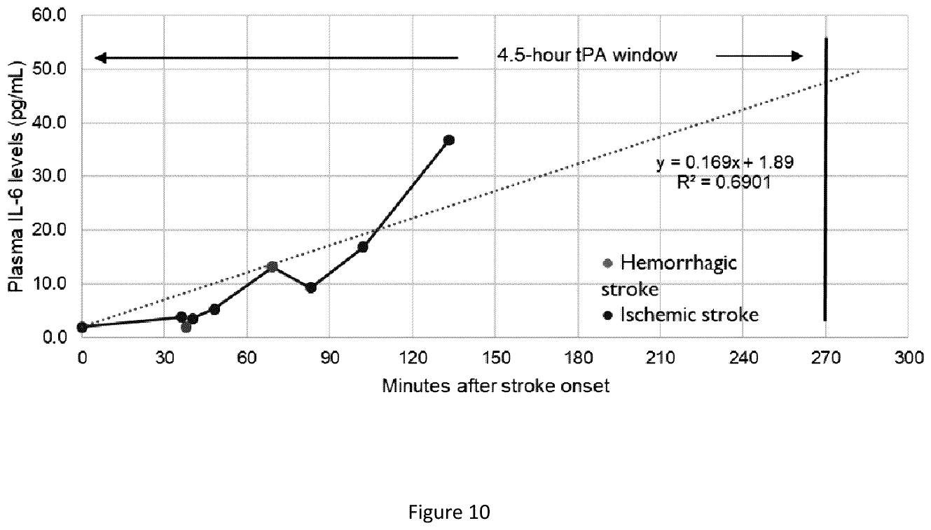

Plasma IL-6 levels were quantified with electrochemiluminescence assay (ECL) in all confirmed stroke patients. The individual IL-6 concentration was elevated (i.e. over 1.89 pg/mL) in all 7 patients with ischemic stroke, and was at the median normal level (i.e. about 1.89 pg/mL) in 1 patient who had a thalamic intracerebral hemorrhage. In the ischemic stroke patients, IL-6 concentrations increased consistently with increased sampling time from symptom onset for individual patients for the blood draw on the MSU: 3.82 pg/mL at 36 minutes, 5.29 pg/mL at 48 minutes, 9.25 pg/mL at 83 minutes, 16.80 pg/mL at 102 minutes and 36.76 pg/mL at 133 minutes (FIG. 2). During the first 133 minutes after stroke onset, plasma IL-6 increased at a rate of 0.4 pg/mL per minute (Table 3, FIG. 2). Median plasma IL-6 concentration was 39.74 pg/mL (range 5.64-94.05) at 24 hours after stroke onset. Median increase during the first 24 hours was 12.96 pg/mL (range 3.88-84.97) (Table 5, FIG. 3). No association was found between plasma IL-6 concentration and time of day blood drawn, initial NIHSS score or patient age (FIG. 7). In ECL analyses, IL-10 and IL-12p70 remained at normal levels acutely and during the next 24 hours. Without wishing to be limited by theory, Applicants surmise that the IL-10 and IL-12p70 response may occur later in the stroke course, and/or is not as robust as the IL-6 increase.

Plasma IL-6 was also evaluated with enzyme-linked immunoassay (ELISA) in the 7 confirmed acute stroke patients (5 AIS, 2 ICH) and 5 healthy adult control subjects. Median age of healthy controls was 70 years (range 65-86) and 3 (60%) were female. In the ELISA analysis, median plasma IL-6 was 7.36 pg/mL (range 2.03-41.05) for stroke patients vs. 2.30 pg/mL (range 0.93-6.22) for healthy controls. In 5 (71%) stroke patients, plasma IL-6 for blood obtained on the MSU was higher than in healthy controls, and in all stroke patients IL-6 was higher in blood obtained on the MSU than the median normal level for healthy adults12 (Table 6).

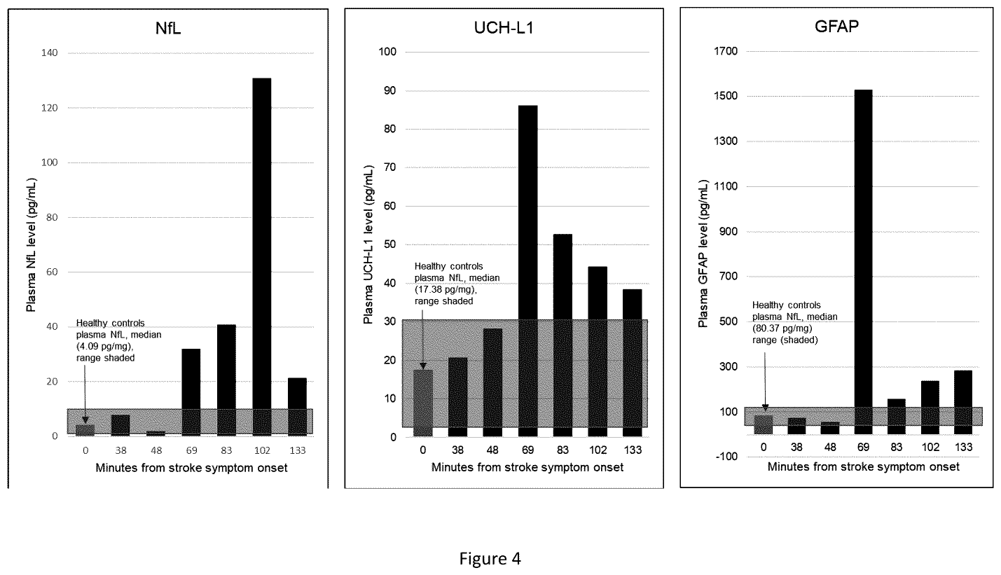

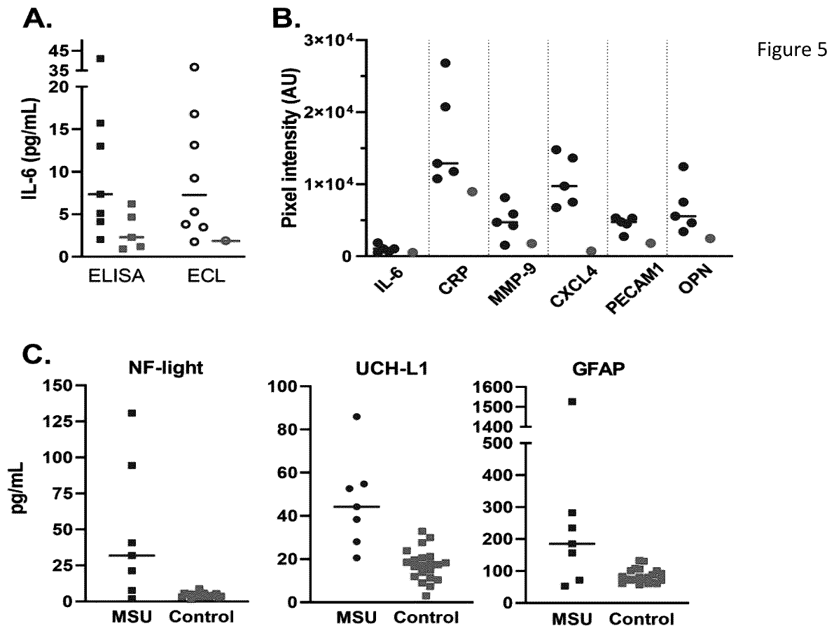

In single molecule array (SIMOA) analysis in 6 confirmed stroke patients median NfL was 26.63 pg/mL (range 1.94-130.82) for stroke patients compared to median 4.09 pg/mL (range 1.34-8.84) for controls. Median UCH-L1 for stroke patients was 41.27 pg/mL (range 20-54-85.98) compared with median 17.38 pg/mL (range 2.98-32.91) for controls. Median GFAP concentration for stroke patients was 195.22 pg/mL (range 52.77-1,526.74) compared to median 80.37 pg/mL (range 56.43-132.86) for healthy controls. In stroke patients, median change in plasma protein levels from symptom onset to 24 hours after stroke was increased for NfL and GFAP, and decreased for UCH-L1. For NfL, levels between stroke onset and 24 hours increased in 3 (60%) patients; for UCH-L1, levels increased in 2 (40%); and for GFAP, levels increased in 5 (100%) patients. (Table 7, FIGS. 4-5).

### Extracellular Vesicle-Derived Neuroinflammatory Factor Levels

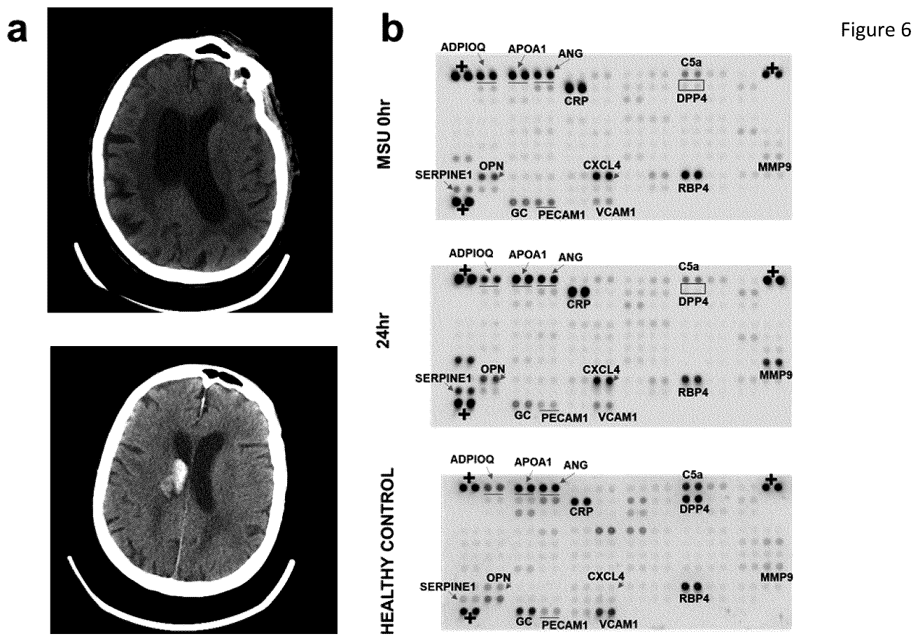

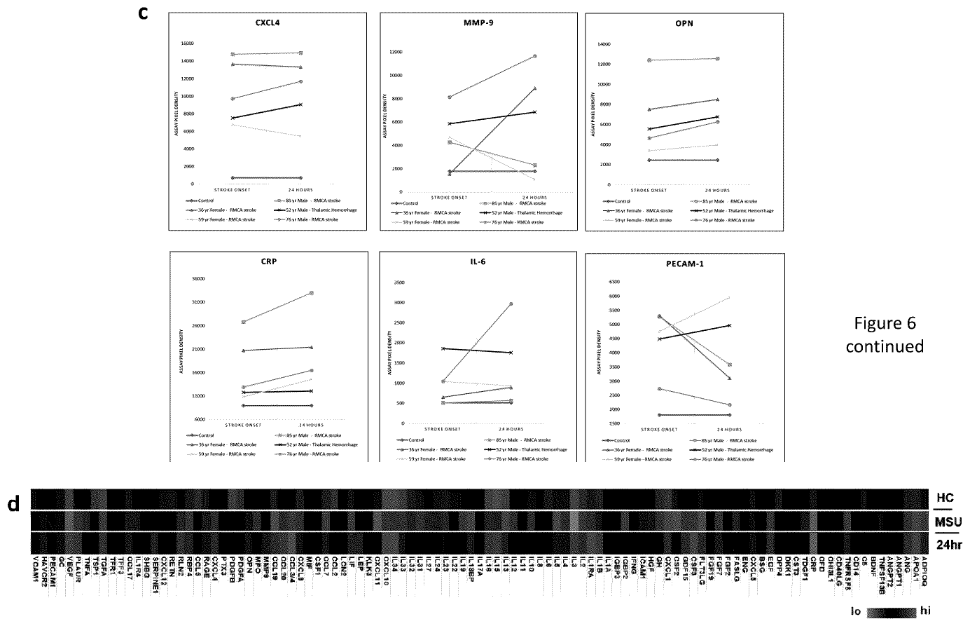

Extracellular vesicles (EV) were isolated from study blood samples with HSP-pull down, and cytokine array analysis was used to quantify EV-derived neuroinflammatory factors in 5 stroke patients (4 AIS, 1 ICH), and 2 pooled samples of blood from healthy adult subjects (FIG. 6). In extracellular vesicle analyses, mean ages of the groups of pooled healthy control subjects were 53±11 years and 54±9.5 years; each group was 50% female. At 24 hours after stroke onset, median levels of all factors increased compared to stroke-onset levels, except for PECAM1. For OPN and CRP, concentrations increased for all patients during this period. For MMP-9, CXCL4, IL-6, and PECAM1, trajectory of change was mixed, with most but not all patient levels increasing over the first 24 hours of stroke progression (FIGS. 6-7).

### Leukocyte Concentrations

White blood cells and subtypes were evaluated in clinical UCH laboratory testing as part of the routine clinical care of MSU patients. Results were available for 7 (87%) of patients with confirmed acute stroke (5 AIS, 2 ICH). Blood for these panels was obtained a median of 116 minutes (range 84-242) after stroke symptom onset. White blood count was elevated in 3 (43%) and normal in 4 (57%). Neutrophils were elevated in 3 (50%) of stroke patients with available values. Monocytes were elevated in 1 (17%) patient. Lymphocytes and eosinophils were normal in all patients (Table 9).

### DISCUSSION

Nine inflammatory factors were fully analyzed, including 6 that reflect neurotoxicity during the hyper-acute phase of stroke (IL-6, neurofilament light chain, GFAP, CRP, MMP-9, CXCL4) and 3 believed to be involved in neuroprotection later in the acute stroke course and recovery (UCH-L1, OPN, PECAM1). Blood samples for all confirmed stroke patients in this report were obtained <133 minutes after stroke onset. To Applicant's knowledge, this represents the earliest the neuroinflammatory cascade has been evaluated in humans after stroke. Furthermore, these results provide a unique snapshot of the neuroinflammatory response in a naturally progressing stroke, before it has been interrupted by treatment. Because inflammatory factors arise from different body systems, the present study attempted to localize neuroinflammatory factors in or on Evs arising from stressed cells displaying surface heat-shock proteins on Evs. This analysis found elevated levels of CRP, MMP-9, CXCL4, PECAM1, IL-6, and OPN, confirming the presence of an active inflammatory process and further refining findings from peripheral blood.

Among the cytokines evaluated in the present study, IL-6 was elevated and selected for further analysis. Previous studies have reported that IL-6 is detected only 4 or more hours after stroke. The present results demonstrated IL-6 was elevated as early as 36 minutes after stroke. Because IL-6 is a systemic cytokine, elevated concentration at stroke onset could represent an acute infection or chronic comorbidity. However, rapidly increasing IL-6 during the hyper-acute (≤2 hours) and acute stage up to 24 hours after stroke suggests an active response to an acute event and, without wishing to be restricted by theory, points to the ischemic or hemorrhagic insult. This theory is bolstered by the lack of significant premorbid disability in any of the stroke patients in this sample (modified Rankin Scale ≤1). IL-6 is of particular interest in stroke as it is measurable from a peripheral blood sample at the site of initial stroke evaluation with point-of-care devices that produce a result in as little as 20 minutes. The present study results demonstrated increasing inflammatory factor levels in all patients with stroke for whom a second blood sample was obtained, during the first 24 hours. This change may demonstrate an active and increasing inflammatory response, which is attributable to the acute stroke, rather than a chronic condition.

Among proteins and chemokines found to be elevated, GFAP is associated with breakdown of the blood-brain barrier in acute brain injury including trauma and stroke. GFAP has shown promise as a biomarker for ischemic/hemorrhagic differentiation in stroke, but thus far with insufficient sensitivity for clinical application. NfL, a component of the neuronal cytoskeleton, reflects neuronal structural insult in stroke. UCH-L1 is a protein associated with brain self repair after stroke, C-reactive protein may be associated with blood-brain barrier disruption, and CXCL4 induces adhesion of neutrophils to cerebral endothelium after acute stroke.

### Example 3—Expanded Study Focusing on IL-6

Forty-two adults were analyzed, including 28 patients treated on the MSU, and 14 controls. Nineteen (68%) MSU patients were diagnosed as stroke, with 17 (61%) confirmed and 2 likely TIA or stroke averted by thrombolysis; 9 were stroke mimics. For confirmed stroke patients, median age was 72 (range 36-87) years, 47% were female, and 82% white. Median initial NIHSS score was 7 (range 1-22) and 12 (71%) received intravenous thrombolysis with tPA or TNK. Blood was obtained a median of 57 (range 26-158) minutes after symptom onset. IL-6 was elevated in all 17 confirmed stroke patients, a median of 3 times normal, as early as 26 minutes after symptom onset (median 6.4 pg/mL).

In 15 (88%) cases, IL-6 increased over 24 hours, (median A 8.9 pg/mL). For mimics and healthy controls, median IL-6 was 5.9 and 2.3 pg/mL, respectively. Initial IL-6 in strokes was not associated with lesion volume (Spearman's r=−0.035, p=0.894) or initial NIHSS (Spearman's r=−0.043, p=0.869). Based on IL-6 trajectories observed, a cutoff for IL-6 set at 8 pg/mL had a sensitivity of 79% and specificity of 36% for estimating the stroke onset time was in the 4.5-hour thrombolysis window.

This study found IL-6 was elevated in all confirmed strokes and increased in a linear fashion during the first 2.5 hours. IL-6 was independent of lesion volume and NIHSS. This suggests a rapid measurement of IL-6 from blood early in acute stroke may estimate onset time. Applicant's findings may aid development of immunomodulatory therapies targeting IL-6 in acute stroke, for example use of an IL-6 inhibitor.

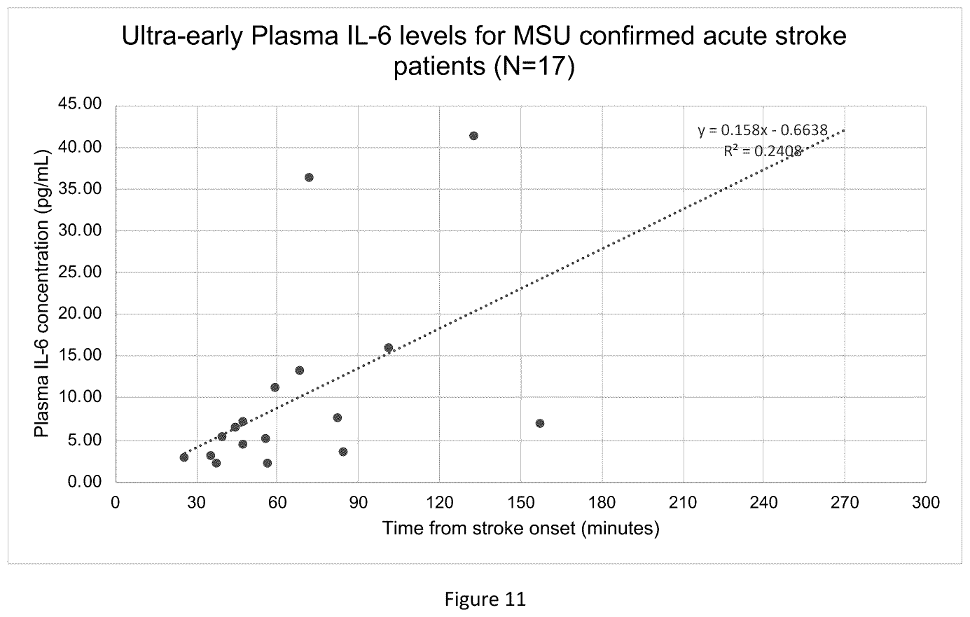

FIG. 11 is a graph showing the individual levels of IL-6 (y axis) measured in blood taken from 17 patients treated on the mobile stroke unit, and confirmed to have had an acute stroke, and the correlation of those IL-6 levels with time from the onset of stroke symptoms (x axis). The trend line is a best-fit line showing increase of IL-6 in circulating blood levels in individuals experiencing acute stroke, with time from stroke symptom onset. All of the IL-6 values were obtained while the patients were in the midst of acute stroke, before treatment was applied.

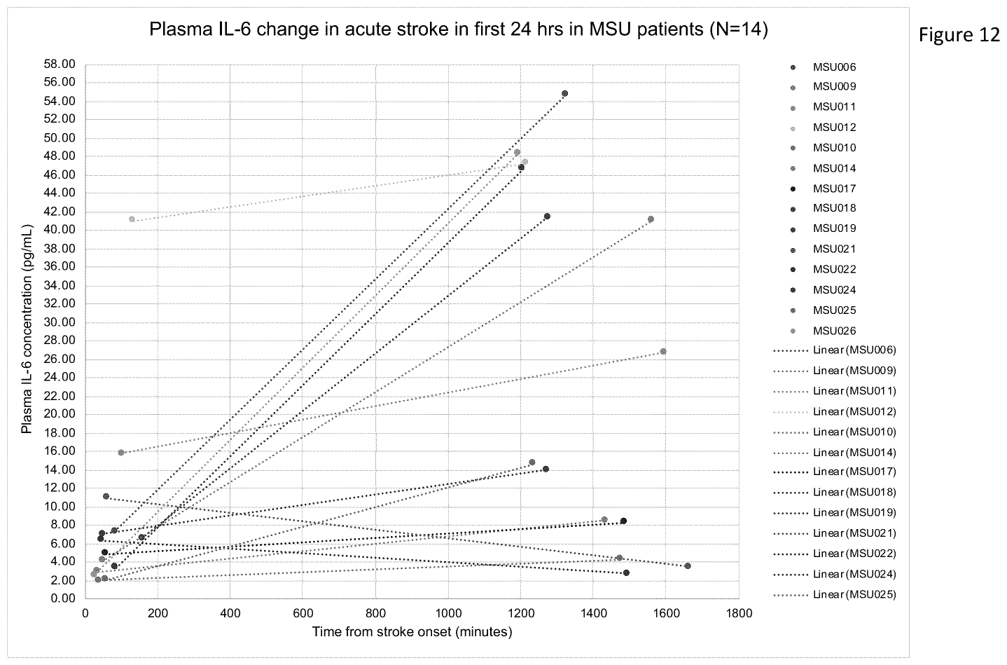

FIG. 12 is a graph showing the individual levels of IL-6 (y axis) at two time points measured in blood taken from 14 patients treated on the mobile stroke unit, and confirmed to have had an acute stroke, and the correlation of those IL-6 levels with time from the onset of stroke symptoms (x axis). The trend lines represent change in IL-6 level for each individual patient between the first IL-6 value measured from blood taken on the mobile stroke unit, and a second blood sample taken at 24±6 hours later in the hospital, typically in the Neuro-ICU.

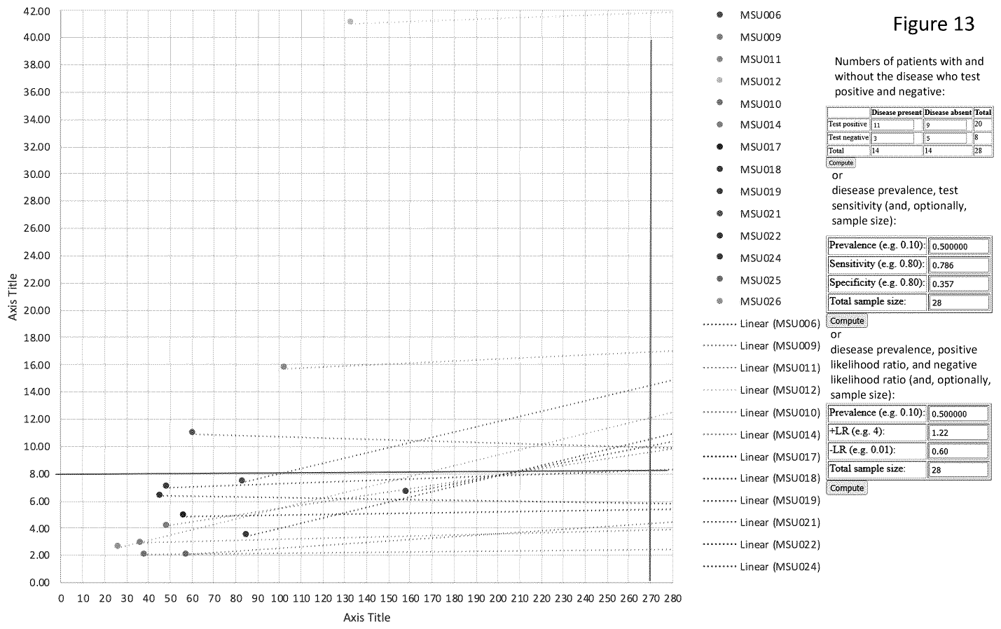

FIG. 13 is the same graph as FIG. 12 resized to focus on the levels of IL-6 at 270 minutes after stroke symptom onset, based on the 24-hour trajectory of change for each patient. The 270 minutes is significant, because this is 4.5 hours after stroke symptom onset, which is the time window for administration of thrombolysis (with tPA or tenecteplase) in acute ischemic stroke. The IL-6 tool we have developed and are refining, estimates when the stroke occurred based on the level of IL-6 measured. The goal is to use this to provide an estimate of when the stroke happened for patients in whom this is not known, and who therefore are not eligible for treatment with thrombolysis. The stroke onset is not known for these patients because it occurred while they were asleep, they are found down alone, or they are found with altered mental status and unable to report when the stroke occurred. This is about 200,000 patients each year in the U.S. who are unable to be treated because the stroke onset time is unknown and there currently is no tool to estimate onset.

FIG. 13 also includes tables showing sensitivity and specificity analysis for estimating whether or not a stroke occurred within 4.5 hours, based on IL-6 values, and cutoff of 8 pg/mL. The Sensitivity in this embodiment is 0.786, and the specificity is 0.357. Thus, the methods, devices, and systems described herein may be able to help make thrombolysis treatment available for, in one embodiment, about half of those patients who would not receive it without the present methods because their stroke onset time is not known. This offers a dramatic improvement in care for 100,000 people each year.

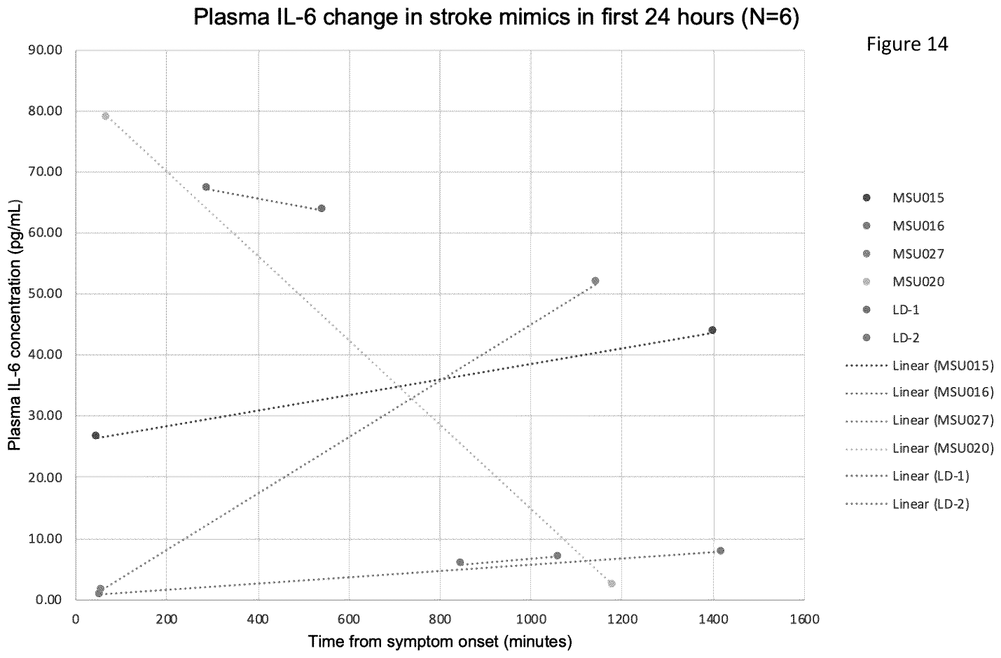

FIG. 14 is a graph showing the change in IL-6 over time for 6 patients who were stroke mimics, that is patients who presented with symptoms consistent with acute stroke but were eventually found to not have stroke.

### REFERENCES

Feigin V L, Stark B A, Johnson C O, Roth G A, Bisignano C, Abady G G, Abbasifard M, Abbasi-Kangevari M, Abd-Allah F, Abedi V, et al. Global, regional, and national burden of stroke and its risk factors, 1990-2019: a systematic analysis for the global burden of disease study 2019. The Lancet Neurology. 2021; 20:795-820.doi: 10.1016/S1474-4422(21)00252-0
Lambertsen K L, Finsen B, Clausen B H. Post-stroke inflammation-tar-get or tool for therapy?. ActaNeuropathol. 2018; 137:693-714. Doi: 10.1007/s00401-018-1930-z Jickling G C, Sharp F R. Biomarker panels in ischemic stroke. Stroke. 2015; 46:915-920. Doi: 10.1161/strokeaha.114.005604
Bustamante A, Penalba A, Orset C, Azurmendi L, Llombart V, Simats A, Pecharroman E, Ventura 0, Ribó M, Vivien D, et al. Blood bio-markers to differentiate ischemic and hemorrhagic strokes. Neurology. 2021;96:e1928-e1939. Doi: 10.1212/wnl.0000000000011742
Luger S, Joger H S, Dixon J, Bohmann F O, Schaefer J, Richieri S P, Larsen K, Hov M R, Bache K G, Foerch C. Diagnostic accuracy of glial fibrillary acidic protein and ubiquitin carboxy-terminal hydrolase-11 serum concentrations for differentiating acute intracerebral hemorrhage from ischemic stroke. Neurocrit Care. 2020; 33:39-48.doi: 10.1007/s12028-020-00931-5
Ren C, Kobeissy F, Alawieh A, Li N, Li N, Zibara K, Zoltewicz S, Guingab-Cagmat J, Lamer S F, Ding Y, et al. Assessment of serum uch-ll and gfap in acute stroke patients. Sci Rep. 2016; 6:24588. Doi: 10.1038/srep24588
Muhammad S, Chaudhry S R, Kahlert U D, Niemela M, Hdnggi D. Brain immune interactions—novel emerging options to treat acute ischemic brain injury. Cells. 2021; 10:2429. Doi: 10.3390/cells10092429
Shichita T, Sugiyama Y, Ooboshi H, Sugimori H, Nakagawa R, Takada I, Iwaki T, Okada Y, Iida M, Cua D J, et al. Pivotal role of cerebral interleukin-17—producing yδt cells in the delayed phase of ischemic brain injury. Nat Med. 2009; 15:946-950. Doi: 10.1038/nm.1999
Ceulemans A-G, Zgavc T, Kooijman R, Hachimi-Idrissi S, Sarre S, Michotte Y. The dual role of the neuroinflammatory response after ischemic stroke: modulatory effects of hypothermia. J Neuroinflammation. 2010; 774. Doi: 10.1186/1742-2094-7-74
Dagonnier M, Donnan G A, Davis S M, Dewey H M, Howells D W. Acute stroke biomarkers: are we there yet?. Front Neurol. 2021; 12:619721.doi: 10.3389/fneur.2021.619721
STROBE reporting guideline. Accessed Nov. 11, 2022. www.Strobe-statement.org/.
Todd J, Simpson P, Estis J, Torres V, Wub A H. Reference range and short- and long-term biological variation of interleukin (il)-6, il-17a and tissue necrosis factor-alpha using high sensitivity assays. Cytokine. 2013; 64:660-665. Doi: 10.1016/j.cyto.2013.09.018
Mayo Clinic Laboratories Plasma Interleukin-6 Clinical Information. Accessed Jun. 8, 2022. www.mayocliniclabs.com/test-catalog/Overview/63020 #Clinical-and-Interpretive.
Nilupul Perera M, Ma H K, Arakawa S, Howells D W, Markus R, Rowe C C, Donnan G A. Inflammation following stroke. J Clin Neurosci. 2006; 13:1-8. Doi: 10.1016/j.jocn.2005.07.005
Amantea D, Nappi G, Bernardi G, Bagetta G, Corasaniti M T. Post-ischemic brain damage: pathophysiology and role of inflammatory mediators. FEBS J. 2009; 276:13-26.doi: 10.1111/j.1742-4658.2008.06766.x
Liu C, Chu D, Kalantar-Zadeh K, George J, Young H A, Liu G. Cytokines: from clinical significance to quantification. Adv Sci. 2021; 8:2004433.doi: 10.1002/advs.202004433
Chaemsaithong P, Romero R, Korzeniewski S J, Martinez-Varea A, Dong Z, Yoon B H, Hassan S S, Chaiworapongsa T, Yeo L. A point of care test for interleukin-6 in amniotic fluid in preterm prelabor rupture of membranes: a step toward the early treatment of acute intra-amniotic 25 inflammation/infec-tion. The Journal of Maternal-Fetal & Neonatal Medicine. 2015; 29:360-367. Doi: 10.3109/14767058.2015.1006621
Fischer S K, Williams K, Wang L, Capio E, Briman M. Development of an il-6 point-of-care assay: utility for real-time monitoring and management of cytokine release syndrome and sepsis. Bioanalysis. 2019; 11:1777-1785. Doi: 10.4155/bio-2019-0192
Bartell P A, Nilsonne G, Lekander M, Akerstedt T, Axelsson J, Ingre M. Diur- nal variation of circulating interleukin-6 in humans: a meta-analysis. PloS One. 2016; 11:e0165799.doi: 10.1371/journal.pone.0165799
Davey M, Benzina S, Savoie M, Breault G, Ghosh A, Ouellette R J. Affinity captured urinary extracellular vesicles provide mrna and mima biomarkers for improved accuracy of prostate cancer detection: a pilot study. Int J Mol Sci. 2020; 21:8330. Doi: 10.3390/ijms21218330
Griffiths S, Cormier M, Clayton A, Doucette A. Differential proteome analysis of extracellular vesicles from breast cancer cell lines by chaperone affinity enrichment. Proteomes. 2017; 5:25. Doi: 10.3390/proteomes5040025
Griffiths S G, Ezrin A, Jackson E, Dewey L, Doucette A A. A robust strategy for proteomic identification of biomarkers of invasive phenotype complexed with extracellular heat shock proteins. Cell Stress Chaperones. 2019; 24:1197-1209.doi: 10.1007/s12192-019-01041-8
Bijnsdorp I V, Maxouri 0, Kardar A, Schelfhorst T, Piersma S R, Pham T V, Vis A, van Moorselaar R J, Jimenez C R. Feasibility of urinary extracellular vesicle proteome profiling using a robust and simple, clinically applicable isolation method. Journal of Extracellular Vesicles. 2017; 6:1313091. Doi: 10.1080/20013078.2017.1313091
Abdelhak A, Foschi M, Abu-Rumeileh S, Yue J K, D′Anna L, Huss A, Oeckl P, Ludolph A C, Kuhle J, Petzold A, et al. Blood gfap as an emerging biomarker in brain and spinal cord disorders. Nat Rev Neurol. 2022; 18:158-172. Doi: 10.1038/s41582-021-00616-3
Amalia L. Glial fibrillary acidic protein (gfap): neuroinflammation biomarker in acute ischemic stroke. Journal of Inflammation Research. 2021; 14:7501-7506.doi: 10.2147/JIR.S342097
Foerch C, Niessner M, Back T, Bauerle M, De Marchis G M, Ferbert A, Grehl H, Hamann G F, Jacobs A, Kastrup A, et al. Diagnostic accuracy of plasma glial fibrillary acidic protein for differentiating intracerebral hemor- rhage and cerebral ischemia in patients with symptoms of acute stroke. Clin Chem. 2012; 58:237-245. Doi: 10.1373/clinchem.2011.172676
Rozanski M, Waldschmidt C, Kunz A, Grittner U, Ebinger M, Wendt M, Winter B, Bollweg K, Villringer K, Fiebach J B, et al. Glial fibrillary acidic protein for prehospital diagnosis of intracerebral hemorrhage. Cerebrovasc Dis. 2017; 43:76-81.doi: 10.1159/000453460
Uphaus T, Bittner S, Groschel S, Steffen F, Muthuraman M, Wasser K, Weber-Kruger M, Zipp F, Wachter R, Groschel K. Nfl (neurofilament light chain) levels as a predictive marker for long-term outcome after ischemic stroke. Stroke. 2019; 50:3077-3084.doi: 10.1161/STROKEAHA.119.026410
Liu H, Povysheva N, Rose M E, Mi Z, Banton J S, Li W, Chen F, Reay D P, Barrionuevo G, Zhang F, et al. Role of uchll in axonal injury and functional recovery after cerebral ischemia. Proc Natl Acad Sci USA. 2019; 116:4643-4650.doi: 10.1073/pnas.1821282116
Kuhlmann C R, Librizzi L, Closhen D, Pflanzner T, Lessmann V, Pietrzik C U, DeCurtis M, Luhmann H J. Mechanisms of c-reactive protein-induced blood-brain barrier dis- ruption. Stroke. 2009; 40:1458-1466. Doi: 10.1161/strokeaha.108.535930
Bustamante A, Simats A, Vilar-Bergua A, Garcia-Berrocoso T, Montaner J. Blood/brain biomarkers of inflammation after stroke and their association with outcome: from c-reactive protein to damage-associated molecular pat- terns. Neurotherapeutics: the journal of the American Society for Experimental NeuroTherapeutics. 2016; 13:671-684.doi: 10.1007/s13311-016-0470-2
Kollikowski A M, Pham M, Marz A G, Papp L, Nieswandt B, Stoll G, Schuhmann M K. Platelet activation and chemokine release are related to local neutrophil- dominant inflammation during hyperacute human stroke. Translational Stroke Research. 2021; 13:364-369. Doi: 10.1007/s12975-021-00938-w.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed description is to be regarded as illustrative in nature and not restrictive.

All references disclosed herein, whether patent or non-patent, are hereby incorporated by reference as if each was included at its citation, in its entirety. In case of conflict between reference and specification, the present specification, including definitions, will control.

Although the present disclosure has been described with a certain degree of particularity, it is understood the disclosure has been made by way of example, and changes in detail or structure may be made without departing from the spirit of the disclosure as defined in the appended claims.

We claim:

1. A method of stroke assessment, comprising:
obtaining a biological sample from a subject suspected of having a stroke;
capturing at least one biomarker from the biological sample;
measuring a level of the at least one biomarker in the biological sample; and
comparing the level of the at least one biomarker to a reference level to determine an occurrence and an onset time of stroke.

2. The method of claim 1, wherein the at least one biomarker is one or more of IL-6, neurofilament light chain (NfL), GFAP, CRP, MMP-9, CXCL4, UCH-L1, OPN, and PECAM1.

3. The method of claim 1 or claim 2, wherein obtaining the biological sample and measuring the level of the at least one biomarker occurs at point of care.

4. The method of any one of claims 1-3, wherein using the level of the at least one biomarker to determine an occurrence and an onset time of the stroke comprises comparing the level to a reference biomarker level, and the biomarker is one or more of IL-6 and NfL, and the measuring is performed by one of ELISA, Ella, SiMoA, ECL, SPR, or molecular array.

5. The method of any one of claims 1-4, further comprising using the onset time to determine whether administering an anti-clotting drug will be effective to treat the stroke.

6. The method of any one of claims 1-5, further comprising administering an anti-clotting drug to the subject if the onset time is determined to be less than or about 4.5 hours prior to the administration time.

7. The method of any one of claims 5-6, wherein the anti-clotting drug comprises tissue plasminogen activator (tPA).

8. A device or kit for identifying a stroke patient, comprising:
a device for receiving a biological sample from a subject suspected of having a stroke;
a device for identifying the presence of least one biomarker in the blood sample obtained from the subject; and
a device for measuring the level of the at least one biomarker in the biological sample.

9. The device or kit of claim 8, wherein the device or kit is a point-of-care device or kit.

10. The device or kit of claim 8 or claim 9, wherein the device or kit is configured to measure the level of the at least one biomarker in less than about 20 minutes after receiving the biological sample.

11. The device or kit of any one of claims 8-10, wherein the at least one biomarker is one or more of IL-6, neurofilament light chain (NfL), GFAP, CRP, MMP-9, CXCL4, UCH-L1, OPN, and PECAM1.

12. The device or kit of any one of claims 8-11, wherein the biological sample is peripheral blood and the at least one biomarker is selected from IL-6 and NfL.

13. A method of stroke assessment, comprising:
obtaining a first biological sample from a subject suspected of having a stroke;
measuring a level of at least one biomarker in the first biological sample;
waiting a period of time;
obtaining a second biological sample from the subject;
measuring a level of at least one biomarker in the second biological sample
comparing the level of the at least one biomarker in the first biological sample to the level of the at least one biomarker in the second biological sample; and
determining an occurrence and an onset time of stroke from the difference in level of the at least one biomarker in the first and second biological sample as a function of the wait time.

14. The method of claim 13, wherein the at least one biomarker is one or more of IL-6, neurofilament light chain (NfL), GFAP, CRP, MMP-9, CXCL4, UCH-L1, OPN, and PECAM1.

15. The method of claim 13 or claim 14, wherein obtaining the biological sample and measuring the level of the at least one biomarker occurs at point of care.

16. The method of any one of claims 13-15, wherein the biomarker is one or more of IL-6 and NfL.

17. The method of any one of claims 13-16, wherein the biomarker is IL-6.

18. The method of any one of claims 13-17, further comprising using the onset time to determine whether administering an anti-clotting drug will be effective to treat the stroke.

19. The method of any one of claims 13-18, further comprising administering an anti-clotting drug to the subject if the onset time is determined to be less than or about 4.5 hours prior to the administration time.

20. The method of any one of claims 18-19, wherein the anti-clotting drug comprises tissue plasminogen activator (tPA).

21. The method of any one of claims 18-20, wherein the biological sample is peripheral blood. cm 21. A method of diagnosing and treating a stroke, the method comprising:
obtaining a blood sample from a subject suspected of having a stroke;
measuring a level of at least one biomarker in the blood sample;
using the level of the at least one biomarker to determine an occurrence and an onset time of the stroke;
determining whether administering an anti-clotting drug will be effective to treat the stroke; and
administering the anti-clotting drug if the onset time is determined to be less than or about 4.5 hours prior, by the measuring the level of the at least one biomarker.

22. A system for determining an occurrence of a stroke comprising:
a device for accepting a first biological sample from a subject suspected of suffering a stroke;
a device for identifying at least one biomarker in the first biological sample;
a device for determining a first concentration of the at least one biomarker;
a device for comparing the concentration to a second concentration; and
a device for displaying an occurrence and a time of onset based on the comparing.

23. The system of claim 22, wherein the second concentration is one of;
a reference level; or
a concentration of the biomarker from a second biological sample collected at a time before the first biological sample was collected.

24. The method of claim 21 or claim 22, wherein the second concentration is a concentration of the biomarker from a second biological sample collected at a time before the first biological sample was collected, and the comparing also determines a stroke onset time.
